SEQUENCE LISTING (9 sequences)

SEQ ID NO: 1 - protein, 19 aa, Artificial Sequence
  G-Protein blocking peptide KB1753
SSRGYYHGIWVGEEGRLSR

SEQ ID NO: 2 - protein, 143 aa, Artificial Sequence
  Anti-Galphai sdAb F11-d2 antibody
EVQLVESGGGLVQPGGSLRLSCEASGFAFSDTWMYWVRQAPGKGLEWVATITRGDQNTYYTESVKGRFTISRDNAKNTLYLQMNSLKSEDTAVYYCAKEGPIGPPDYWGQGTQVTVSSAAAEQKLISEEDLNGAAHHHHHHGS

SEQ ID NO: 3 - protein, 143 aa, Artificial Sequence
  Anti-Galphai adAb F4-d2 antibody
EVQLVESGGGLVQPGGSLRLSCAASGFTFSSYYMNWVRQAPGKGLEWVSSIYNGDGDTDYSDSVKGRFTISRDNAKNTVYLEMSDLKVQDTAIYYCNAYDGTLLRDYWGQGTQVTVSSAAAEQKLISEEDLNGAAHHHHHHGS

SEQ ID NO: 4 - protein, 145 aa, Artificial Sequence
  Anti-Galphai sdAb G6-d2 antibody
EVQLVESGGDLVQPGGSLRLSCAASGFTFSTYRMNWVRQAPGKGLEWVASISSRGITTDYAHPVKGRFTVSRDNTKNTLYLQMNSLKPDDAAVYYCATDPSTWYRGTAHWGQGTQVTVSSAAAEQKLISEEDLNGAAHHHHHHGS

SEQ ID NO: 5 - protein, 143 aa, Artificial Sequence
  Anti-Galphai sdAb B11-d2 antibody
EVQLVESGGGLVQPGGSLRLSCAASGFTFNDYWMYWVRQAPGKGLEWVSSINTGGSSTYYSDSVKGRFTTARDNAKNSLYLQLNSLRPEDTAVYYCAKEGPVGPPDYWGQGTQVTVSSAAAEQKLISEEDLNGAAHHHHHHGS

SEQ ID NO: 6 - protein, 145 aa, Artificial Sequence
  Anti-Galphai sdAb F9-d2 antibody
EVQLVESGGGLVQPGGSLRLSCAASGFTFDDYAMNWVRQAAGKGPEWVASISSRGITTDYAHSVKGRFTVSRDNTKNTLYLQMNSLKPDDAAVYYCATDPSTWYRGTAHWGQGTQVTVSSAAAEQKLISEEDLNGAAHHHHHHGS

SEQ ID NO: 7 - protein, 148 aa, Artificial Sequence
  Anti-Galpha sdAb G7-d2 antibody
QVQLQESGGGLVQPGGSLRLSCAASGFTFSWYGMSWVRQAPGKGPEWVASISSRGITTDYAHSVKGRFTVSRDNTKNTLYLQMNSLKPEDTGVYYCTRVLWYDSGAYSLNDFWGQGTQVTVSSAAAEQKLISEEDLNGAAHHHHHHGS

SEQ ID NO: 8 - protein, 142 aa, Artificial Sequence
  Anti-Galphai sdAb A10-d2 antibody
EVQLVESGGGLVQPGGSLRLSCAASGLTYSDYAMSWVRQAAGKGPEWVASISSRGITTDYAHSVKGRFTVSRDNTKNTLYLQMNSLEPEDTAVYYCTTVSYWRYEYWGQGTQVTVSSAAAEQKLISEEDLNGAAHHHHHHGS

SEQ ID NO: 9 - protein, 148 aa, Artificial Sequence
  Anti-Galphai sdAb E2-d2 antibody
EVQLVESGGGLVQPGGSLRLSCIGSGYTFSSYAMAWVRQASGKGPEWVASISSRGITTDYAHSVKGRFTISRDNAKNTLYLHMNNLKPEDTALYYCTRVLWYENGLYSLNDFWGQGTQVTVSSAAAEQKLISEEDLNGAAHHHHHHGS

(19) United States
(12) Patent Application Publication
(10) Pub. No.: US 2023/0135945 A1
(43) Pub. Date: May 4, 2023

(54) Method for Measuring Modulation in the Activity of a G Protein-Coupled Receptor

(71) Applicants: CISBIO BIOASSAYS, Codolet (FR); CENTRE NATIONAL DE LA RECHERCHE SCIENTIFIQUE, Paris (FR)

(72) Inventors: Thomas Fabien Michel ROUX, Nimes (FR); Mélanie DA SILVA, Saint-Macoux (FR); Elodie Julie DUPUIS, Caissargues (FR); Eric Jacques Christian TRINQUET, Pont-Saint-Esprit (FR); Julien Jean-Marius SOULE, Montbazin (FR); Philippe RONDARD, Saint-Gely-Du-Fesc (FR); Jean-Philippe R. PIN, Montpellier (FR)

(21) Appl. No.: 16/634,830

(22) PCT Filed: Jul. 27, 2018
(86) PCT No.: PCT/FR2018/051948

§ 371 Date: Jan. 28, 2020

(30) Foreign Application Priority Data
Jul. 28, 2017 (FR) 1757263

(51) Int. Cl. G01N 33/68; G01N 33/542

(52) U.S. Cl. CPC G01N 33/6872; G01N 33/542; G01N 2333/726; G01N 2500/02; G01N 2333/4719

(57) ABSTRACT

The invention relates to a method for determining the ability of a molecule to modulate the activity of a G protein-coupled receptor (GPCR), said method comprising the following steps: a) introducing, into a first container:—a first membrane preparation bearing one or more GPCRs and one or more G protein(s),—a source of GDP or a source of GTP,—a first ligand of the alpha subunit of a G protein (Galpha protein) labelled with a first member of a pair of RET partners, and a second ligand of the Galpha protein labelled with a second member of the pair of RET partners, said ligands being able to specifically bind, either individually or in combination, to the empty Galpha protein or to the full Galpha protein; b) measuring the RET signal emitted in the first container; c) introducing (i) into a second container, the same reactants as in step a) and the molecule to be tested or (ii) into the first container, the molecule to be tested; d) measuring the RET signal emitted in the second container or in the first container obtained in step c); e) comparing the signals obtained in steps b) and d).

| | |
|---|---|
| AA | GPCR activation |
| BB | from “full” G to “empty” G form |
| CC | Agonist |
| DD | Format 1 |
| EE | Format 2 |
| FF | G_{empty} |
| GG | Before activation |
| HH | After activation |

### TECHNICAL FIELD

The invention relates to a novel process for measuring modulation of G-protein-coupled receptor (GPCR) activation, for example a process for determining the ability of a molecule to modulate GPCR activation. The process according to the invention makes it possible to detect the appearance or disappearance of an empty G protein or a full G protein in a GPCR preparation.

### PRIOR ART

G-protein-coupled receptors (GPCRs) are a family of membrane receptors in mammals and throughout the animal kingdom. G proteins are heterotrimeric proteins (3 subunits: alpha, beta and gamma) that are activated by GPCRs. Through GPCRs, G proteins have a role in transducing a signal from outside the cell to inside the cell (i.e. cellular response to an external stimulus). Their commonly described mechanism of action is presented in FIG. 1 and summarized below:
in its inactive state, resting state, the alpha subunit of the G protein is bound to the nucleotide GDP (full G protein bound to GDP);
after activation of the GPCR, the GPCR binds to the alpha subunit of the G protein and triggers a G protein activation process consisting of two steps: 1) exiting the GDP of the G protein to give an empty G protein, and forming an inactive GPCR/empty G protein complex, and 2) binding the GTP which leads to the formation of an active G protein, in GTP form (full G protein bound to GTP). In the first step, the G protein bound to the receptor is in a form called “empty form”. This state is described in the literature as transient since it is described that the nucleotide GTP binds rapidly to the alpha subunit of the G protein. In addition, the beta/gamma subunits of the activated G protein dissociate from the alpha subunit;
the alpha subunit of the full G protein bound to GTP then binds to the effectors to activate them. The effectors in turn activate signaling pathways leading to a cellular response;
GTP is then hydrolyzed to GDP by the G protein alpha subunit and the alpha subunit reunites with the beta/gamma subunit to reform the full G protein bound to GDP (inactive state).

There are several subtypes of G alpha proteins with different selectivity profiles for the different effectors and thus inducing the activation of preferential signaling pathways. GPCRs are associated with many important physiological functions and are considered one of the preferred therapeutic targets for a wide range of diseases. Thus, many in vitro screening tests have been developed to identify molecules capable of modulating GPCRs. The tests developed exploit different mechanisms of G protein activation and implement various technologies (Zhang et al.; Tools for GPCR drug discovery; (2012) Acta Pharmacologica Sinica).

Particular mention may be made of affinity tests that use radiolabeled ligands to measure the affinity of the ligand to the GPCR, proximity scintigraphy tests that use scintigraphy beads to which GPCRs have been attached or functional tests using slowly or non-hydrolyzable GTP such as GTPyS. However, these tests are difficult to implement and sometimes require membrane filtration steps that may limit their use as high-throughput screening (HTS) assays.

Other assays have been developed to demonstrate GPCR activation. These tests are based in particular on resonance energy transfer (RET) techniques such as fluorescence resonance energy transfer (FRET) or bioluminescence resonance energy transfer (BRET). Examples include energy transfer techniques that highlight the interaction between a GPCR and G protein by using either a donor fused to the GPCR and an acceptor fused to G protein (WO 2006/086883 and WO 2003/008435) or an acceptor fused to the G protein alpha subunit and a donor fused to the G protein beta and/or gamma subunit (Bunemann et al. Proc. Natl. Acad. Sci., 2003, 26, 16077-16082). However, these techniques are constraining since they require the preparation of fusion proteins and do not allow the study of GPCRs and G proteins expressed endogenously by cells (i.e. unmodified and not overexpressed). On the other hand, in order to discriminate between the different subtypes of G alpha proteins that can be activated by the receptor, these techniques require the preparation of multiple membrane samples (a specific preparation for each subtype of G alpha protein).

Energy transfer techniques have also been used to develop tests to visualize the modulation of the GTP (active) form of the G protein or the GDP (inactive) form of the G protein. One example is the use of a format with the G protein fused to a biotin label thus bound to a donor coupled to a streptavidin protein and an acceptor bound to a non-hydrolyzable or slowly hydrolyzable GTP analogue (WO 2006/035208). On the other hand, another format uses BioKey® biotinylated peptides (Karo Bio) that discriminate between the GTP and GDP forms and are bound to a donor through a streptavidin protein coupled to the donor. The acceptor is bound to the GPCR, which is fused with a 6HIS tag, using an anti-6HIS antibody (WO 2004/035614). These techniques are also constraining since they also require the preparation of fusion proteins and do not allow the study of GPCRs and G proteins expressed endogenously by cells. Similarly, in order to discriminate between the different subtypes of G alpha proteins that can be activated by the receptor, these techniques require the preparation of multiple membrane samples.

Finally, a last energy transfer format uses the biotinylated BioKey® peptides (Karo Bio) described above with streptavidin coupled to a donor and a GTPyS analogue bound to an acceptor (Frang et al., https://shop.perkinelmer.com/Content/relatedmaterials/posters/sps_006943gtplance.pdf). However, this last format does not make it possible to distinguish the form of the non-associated G protein (G protein bound to a GDP or GTP nucleotide) from the form associated with the GPCR when it is activated by a compound (empty G protein).

There is therefore a real need for a sensitive and reliable method to easily determine the modulation of GPCR activation, for example to easily determine the ability of a molecule to modulate GPCR activation, and/or to determine which G protein subtype is activated by the GPCR.

### SUMMARY OF THE INVENTION

The present invention aims to propose a novel method for the in vitro screening of molecules capable of modulating GPCRs. This novel method is notably based on the ability to discriminate between a full form of G protein (full G protein bound to GTP or full G protein bound to GDP) and an empty form of G protein, an approach that, to the knowledge of the inventors, has never been used to measure the activation of a GPCR.

In particular, the present invention has the advantages of 1) using a fluorescence-based detection method, which is therefore non-radioactive; 2) not requiring any washing steps and thus simplifying its implementation, particularly for screening activities for high-throughput compounds; 3) allowing work in particular on unmodified G and GPCR proteins; 4) allowing the discrimination of different subtypes of Galpha proteins activated by the GPCR in the same membrane preparation containing these different subtypes (the discrimination being provided by the use of detection ligands discriminating against Galpha protein subtypes).

According to a first aspect, the invention relates to a method for determining the ability of a molecule to modulate the activation of a G-protein-coupled receptor (GPCR), said method comprising the following steps:
a) introduction, into a first container, of:
   a membrane preparation carrying one or more GPCRs and one or more G proteins,
   a GDP source or a GTP source,
   a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
b) measurement of the RET signal emitted in the first container;
c) introduction (i) into a second container of the same reagents as in step a) and the test molecule or (ii) into the first container of the test molecule;
d) measurement of the RET signal emitted in the second container or in the first container obtained in step c);
e) comparison of the signals obtained in steps b) and d);
   an increase in signal when using a GDP source and ligands capable of binding specifically to the empty Galpha protein indicating that the molecule is a GPCR agonist;
   a decrease in signal when using a GDP source and ligands capable of binding specifically to the empty Galpha protein indicating that the molecule is a GPCR inverse agonist;
   a decrease in signal when using a GDP source and ligands capable of binding specifically to the full Galpha protein indicating that the molecule is a GPCR agonist;
   an increase in signal when using a GDP source and ligands capable of binding specifically to the full Galpha protein indicating that the molecule is a GPCR inverse agonist;
   an increase in signal when using a source of GTP and ligands capable of binding specifically to the empty Galpha protein indicating that the molecule is a GPCR agonist;
   a decrease in signal when using a source of GTP and ligands capable of binding specifically to the empty Galpha protein indicating that the molecule is a GPCR inverse agonist;
   a decrease in signal when using a source of GTP and ligands capable of binding specifically to the full Galpha protein indicating that the molecule is a GPCR agonist;
   an increase in signal when using a source of GTP and ligands capable of binding specifically to the full Galpha protein indicating that the molecule is a GPCR inverse agonist.

According to a second aspect, the invention relates to a method for determining the ability of a molecule to modulate the activation of a G-protein-coupled receptor (GPCR), said method comprising the following steps:
a) introduction, into a first container, of:
   a membrane preparation carrying one or more GPCRs and one or more G proteins,
   a GDP source or a GTP source,
   a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
   a GPCR agonist;
b) measurement of the RET signal emitted in the first container;
c) introduction into a second container of the same reagents as in step a) and of the test molecule;
d) measurement of the RET signal emitted in the second container obtained in step c);
e) comparison of the signals obtained in steps b) and d);
   an increase in signal when using a GDP source and ligands capable of binding specifically to the empty Galpha protein indicating that the molecule is a GPCR agonist or positive allosteric modulator;
   a decrease in signal when using a GDP source and ligands capable of binding specifically to the full Galpha protein indicating that the molecule is a GPCR agonist or positive allosteric modulator;
   an increase in signal when using a source of GTP and ligands capable of binding specifically to the empty Galpha protein indicating that the molecule is a GPCR agonist or positive allosteric modulator;
   a decrease in signal when using a source of GTP and ligands capable of binding specifically to the full Galpha protein indicating that the molecule is a GPCR agonist or positive allosteric modulator.
   a decrease in signal when using a GDP source and ligands capable of binding specifically to the empty Galpha protein indicating that the molecule is a GPCR antagonist or negative allosteric modulator;
   an increase in signal when using a GDP source and ligands capable of binding specifically to the full Galpha protein indicating that the molecule is a GPCR antagonist or negative allosteric modulator;
   a decrease in signal when using a source of GTP and ligands capable of binding specifically to the empty Galpha protein indicating that the molecule is a GPCR antagonist or negative allosteric modulator;
   an increase in signal when using a source of GTP and ligands capable of binding specifically to the full Galpha protein indicating that the molecule is a GPCR antagonist or negative allosteric modulator.

According to a third aspect, the invention relates to a kit for implementing the method according to the invention comprising:
a first ligand of the G protein alpha subunit (Galpha protein) labeled with a first member of a RET partner pair and a second ligand of the Galpha protein labeled with a second member of the RET partner pair, said ligands being capable of binding specifically, either individually or in combination, to the empty Galpha protein or to the full Galpha protein; and
a set of instructions;
optionally one or more dilution buffers for the reagents;
optionally a GDP source or a GTP source;
optionally a membrane preparation carrying one or more GPCRs and/or one or more proteins; and
optionally a Galpha protein.

### BRIEF DESCRIPTION OF THE FIGURES

FIG. 1 illustrates the mechanism of action of GPCR and G protein activation.

FIG. 2 illustrates the concept of detection of the activation of the GPCR and of the associated G protein by discriminating between an initial form of full G protein (bound to GDP or GTP or non-hydrolyzable/slowly hydrolyzable GTP) and a final empty form (without nucleotide).

FIGS. 3A to 3D illustrate 4 test formats according to the invention.

FIGS. 4A and 4B illustrate an activation test according to format 2B with the detection ligand pair: Biotin Peptide-KB1753/SA-XL+anti Galphai antibody SC13533-Lumi4Tb.

FIGS. 5A and 5B illustrate an activation test according to format 2B with the detection ligand pair: anti Galphai DSV36S-d2+anti Galphai SC13533-Lumi4Tb antibody.

FIGS. 6A and 6B illustrate an activation test according to format 2B with the detection ligand pair: anti Galphai DSV39S-d2+anti Galphai SC13533-Lumi4Tb antibody.

FIGS. 7A and 7B illustrate an activation test according to format 2B with the detection ligand pair: anti Galphai DSV3S-d2+anti Galphai DSV36S-Lumi4Tb antibody.

FIGS. 8A and 8B illustrate an activation test according to format 1A with the detection ligand pair: anti-Galphai antibody DSV39S-d2+anti-Galphai antibody DSV26S-Lumi4Tb.

FIGS. 9A and 9B illustrate an activation test according to format 2A with the detection ligand pair: anti Galphai DSV39S-d2 +anti Galphai DSV26S-Lumi4Tb antibody.

FIG. 10 illustrates an activation test according to format 2B with the detection ligand pair: Biotin-KB1753/SA-XL peptide+anti Galphai antibody SC13533-Lumi4Tb, with validation of the test format on four different GPCRs (Delta Opioid, GalaninR1, NOP, 5HT1A) and on two different cell grounds (HEK293 and CHO-K1).

FIG. 11 illustrates an activation test according to format 2A with the detection ligand pair: anti Galphai DSV39S-d2+anti Galphai DSV26S-Lumi4Tb antibody, with validation of the test format with GTPγS or GTP.

FIGS. 12A and 12B illustrate an activation test according to format 2B with the detection ligand pair: DSV36S-Lumi4Tb anti-Galphai antibody+SC13533-d2 anti-Galphai antibody.

FIGS. 13A and 13B illustrate an activation test according to format 2B with the detection ligand pair: anti Galphai sdAb F11-d2 antibody+anti Galphai SC13533-Lumi4Tb antibody.

FIGS. 14A and 14B illustrate an activation test according to format 2B with the detection ligand pair: anti Galphai sdAb F4-d2 antibody+anti Galphai SC13533-Lumi4Tb antibody.

FIGS. 15A and 15B illustrate an activation test according to format 2B with the detection ligand pair: anti Galphai sdAb G6-d2 antibody+anti Galphai SC13533-Lumi4Tb antibody.

FIGS. 16A and 16B illustrate an activation test according to format 2B with the detection ligand pair: anti Galphai sdAb B11-d2 antibody+anti Galphai SC13533-Lumi4Tb antibody.

FIGS. 17A and 17B illustrate an activation test according to format 2B with the detection ligand pair: anti Galphai sdAb F9-d2 antibody +anti Galphai SC13533-Lumi4Tb antibody.

FIGS. 18A and 18B illustrate an activation test according to format 2B with the detection ligand pair: anti Galphai sdAb G7-d2 antibody+anti Galphai SC13533-Lumi4Tb antibody.

FIGS. 19A and 19B illustrate an activation test according to format 2B with the detection ligand pair: anti Galphai sdAb A10-d2 antibody+anti Galphai DSV36S-Lumi4Tb antibody.

FIG. 20A illustrates the ability of the anti Galphai sdAb E2-d2+anti-Galphai SC13533-Lumi4Tb detection ligand pair to specifically detect the full GTPgS form of the Galpha protein from the empty form of the Galpha protein.

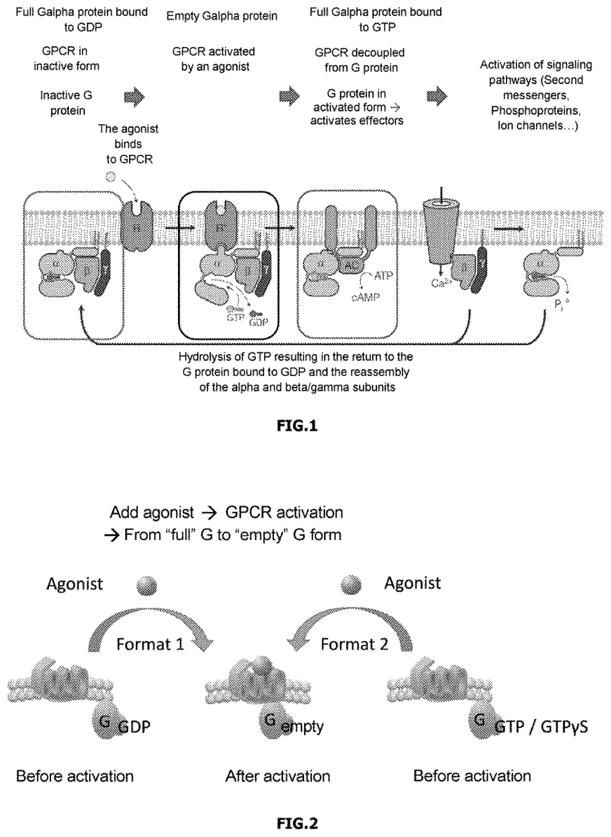

### DETAILED DESCRIPTION OF THE INVENTION

Definitions

In the sense of the invention, the term “G protein” refers to a heterotrimeric protein composed of three subunits called Galpha protein, Gbeta protein and Ggamma protein. In the sense of the invention, the term “Galpha protein” or “Galpha” refers to the G protein alpha subunit. The Galpha protein has two domains, the GTPase domain, and the alpha helix domain. There are at least 20 different Galpha proteins, which can be classified into the following main protein families: Galphas (known to activate adenylate cyclase to increase cAMP synthesis), Galphai (known to inhibit adenylate cyclase), Galphaolf (associated with olfactory receptors), Galphat (known for transducing visual signals in the retina in conjunction with rhodopsin), Galphaq (known to stimulate phospholipase C) or the Galpha12/13 family (known to regulate cytoskeleton, cell junctions, and other processes related to cell movement). In a preferred embodiment of the invention, the Galpha protein is selected from the Galphai1, Galphai2, Galphai3, Galphao1, Galphao2, Galphaq, Galpha12, Galpha13, Galphas, Galphaz, Galphat1, Galphat2, Galpha11, Galpha14, Galpha15, Galpha16 and Galphagus protein, preferably selected from the Galphai1, Galphai2 and Galphai3 protein.

In the sense of the invention, the term “full Galpha protein” refers to a Galpha protein bound to GTP or to GTP that is non-hydrolyzable or slowly hydrolyzable or to GDP. This term therefore refers to both the Galpha protein bound to GDP (“GDP-bound full Galpha protein”) and the Galpha protein bound to GTP or to non-hydrolyzable or slowly hydrolyzable GTP (“GTP-bound full Galpha protein”). The full Galpha protein (bound to GDP or GTP) is shown in FIG. 1.

The term “GDP” refers to guanosine diphosphate.

The term “GTP” refers to guanosine triphosphate.

The term “GTP source” refers to a compound that provides GTP and/or non-hydrolysable or slowly hydrolysable GTP, for example GTP as such and/or non-hydrolysable or slowly hydrolysable GTP as such.

The term “GDP source” refers to a compound that provides GDP, for example GDP as such.

The term “non-hydrolyzable or slowly hydrolyzable GTP” refers to an analogue of GTP that is not hydrolyzed or slightly hydrolyzed to GDP. Examples include GTPgammaS (CAS no. 37589-80-3), GppNHp (CAS no. 148892-91-5) or GppCp (CAS no. 10470-57-2). The terms “GTPgS” or “GTPγS” are abbreviations of the term “GTPgammaS”.

In the sense of the invention, the term “empty Galpha protein” refers to a Galpha protein that is not bound to GTP or to GDP or to non-hydrolyzable or slowly hydrolyzable GTP. The empty Galpha protein is described in the literature as a transitional state between the GDP-bound full form and the full form bound to GTP or to non-hydrolyzable or slowly hydrolyzable GTP. The empty Galpha protein is shown in FIG. 1.

In the sense of the invention, the term “membrane preparation” refers to a preparation comprising cell membranes or fragments of cell membranes or artificial systems mimicking cell membranes that carry (or express on their surface) one or more GPCRs and one or more Galpha proteins. Thus, the term “membrane preparation” includes whole cells, permeabilized whole cells, lysed cells, purified cell membranes and purified GPCR/Galpha protein complexes purified and reconstituted in nanodiscs (also called “nanoscale phospholipid bilayers”) or detergent mixtures that carry (or express on their surface) one or more GPCRs and one or more Galpha proteins.

An “antibody” according to the invention may be of mammalian origin (e.g. human or mouse or camelid), humanized, chimeric, recombinant. It is preferably a monoclonal antibody produced recombinantly by genetically modified cells using techniques widely known to the skilled person. The antibody can be of any isotype, e.g. IgG, IgM, IgA, IgD or IgE.

An “antibody fragment” according to the invention can for example be selected from fragments Fv, Fab, Fab′, Fab′-SH, F(ab′)², diabodies, single-domain antibodies, single-chain antibodies (e.g. scFv).

The terms “single-domain antibody and “sdAb” are interchangeable and refer to an antibody in which binding to the antigen is performed by a single variable domain. An sdAb may be i) an antibody comprising or consisting of a heavy chain variable domain (HV) or a fragment thereof capable of binding to the antigen, which binds to the antigen independently of any other variable domain, ii) an antibody comprising or consisting of a light chain variable domain (LV) or a fragment thereof capable of binding to the antigen, which binds to the antigen independently of any other variable domain, or ii) an antibody comprising or consisting of a heavy chain variable domain of the V_{H}H type (V_{H}H) or a fragment thereof capable of binding to the antigen, which binds to the antigen independently of any other variable domain.

In the sense of the invention, the terms “ligand capable of binding specifically to the empty form” and “ligand capable of binding specifically to the full form” refer respectively to a ligand capable of binding preferentially to the empty form or to the full form of G protein. That is to say a ligand having respectively a better affinity (i.e. a smaller Kd) for the empty form or the full form of G protein with respect to the full form or the empty form of G protein, respectively. The selectivity factor (ratio of Kd for the preferentially recognized form to Kd for the other form) is, for example, at least a factor of two.

In the sense of the invention, the terms “ligands capable of binding specifically, in combination, to the empty Galpha protein” and “ligands capable of binding specifically, in combination, to the full Galpha protein” mean respectively:
1) a pair of ligands capable of preferentially binding to an empty form of the Galpha protein, i.e. a pair of ligands of which at least one of the two ligands has, alone or in combination with the second ligand, a better affinity (i.e. a smaller Kd) for the empty form of the Galpha protein with respect to the full form of the Galpha protein. The selectivity factor (ratio of Kd for the empty form to Kd for the full form) is, for example, at least a factor of two.
2) a pair of ligands capable of preferentially binding to a full form of the Galpha protein, i.e. a pair of ligands of which at least one of the two ligands has, alone or in combination with the second ligand, a better affinity (i.e. a smaller Kd) for the full form of the Galpha protein with respect to the empty form of the Galpha protein. The selectivity factor (ratio of Kd for the full form to Kd for the empty form) is, for example, at least a factor of two.

It may also be a pair of ligands capable of generating a higher RET signal with one form of the Galpha protein versus the other form of the Galpha protein. The RET signal ratio (ratio of the RET signal for the preferentially recognized form to the RET signal for the other form) is, for example, at least a factor of 1.3.

In the sense of the invention, the term “molecule capable of modulating GPCR activation” refers to a molecule capable of activating or inhibiting a GPCR, and thus inducing transduction or preventing the transduction of a signal from outside the cell to inside the cell via the GPCR. It may be an agonist, an antagonist, a reverse agonist, a positive allosteric modulator or a negative allosteric modulator.

In the sense of the invention, the term “test molecule” is a molecule capable of modulating GPCR activation.

In this description, the term “molecule”, without further clarification, refers to both “molecule capable of modulating GPCR activation” and “test molecule”.

The term “RET” refers to resonance energy transfer.

The term “FRET” refers to fluorescence resonance energy transfer. FRET is defined as a non-radiative energy transfer resulting from a dipole-dipole interaction between a donor and an energy acceptor. This physical phenomenon requires an energetic compatibility between these molecules. This means that the donor's emission spectrum must cover, at least partially, the acceptor's absorption spectrum. In accordance with Förster's theory, FRET is a process that depends on the distance between the two molecules, donor and acceptor: when these molecules are in proximity to each other, a FRET signal will be emitted.

The term “BRET” refers to bioluminescence resonance energy transfer.

In the sense of the invention, the term “ligand” refers to a molecule capable of binding specifically and reversibly to a target molecule. In the context of the invention, the target molecule is the empty Galpha protein or the full Galpha protein. The ligand can be of a protein nature (e.g. a protein or peptide) or of a nucleotide nature (e.g. DNA or RNA). In the context of the invention, each ligand is advantageously selected from an antibody, an antibody fragment, a peptide or an aptamer.

In the sense of the invention, the ligand is labeled with a member of a RET partner pair. The ligand may be labeled directly or indirectly by methods well known to the skilled person, for example as described below, but preferably the ligand is labeled directly, by covalent bonding with a member of a RET partner pair.

The term “RET partner pair” refers to a pair consisting of an energy donor compound (hereinafter referred to as the “donor compound”) and an energy acceptor compound (hereinafter referred to as the “acceptor compound”); when they are in proximity to each other and excited at the excitation wavelength of the donor compound, these compounds emit a RET signal. It is known that for two compounds to be RET partners, the emission spectrum of the donor compound must partially cover the excitation spectrum of the acceptor compound. For example, “FRET partner pairs” is used when using a fluorescent donor compound and an acceptor compound or “BRET partner pair” is used when using a donor bioluminescent compound and an acceptor compound.

The term “RET signal” refers to any measurable signal representative of a RET between a donor compound and an acceptor compound. For example, a FRET signal may therefore be a variation in the intensity or lifetime of luminescence of the fluorescent donor compound or the acceptor compound when the latter is fluorescent.

The term “container” refers to a well of a plate, a test tube or other suitable container for mixing a membrane preparation with the reagents necessary for the implementation of the method according to the invention.

Methods

According to a first aspect, the invention relates to a method for determining the ability of a molecule to modulate the activation of a G-protein-coupled receptor (GPCR), said method comprising the following steps:
a) introduction, into a first container, of:
   a membrane preparation carrying one or more GPCRs and one or more G proteins,
   a GDP source or a GTP source,
   a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
b) measurement of the RET signal emitted in the first container;
c) introduction (i) into a second container of the same reagents as in step a) and the test molecule or (ii) into the first container of the test molecule;
d) measurement of the RET signal emitted in the second container or in the first container obtained in step c);
e) comparison of the signals obtained in steps b) and d);
   an increase in signal when using a GDP source and ligands capable of binding specifically to the empty Galpha protein indicating that the molecule is a GPCR agonist;
   a decrease in signal when using a GDP source and ligands capable of binding specifically to the empty Galpha protein indicating that the molecule is a GPCR inverse agonist;
   a decrease in signal when using a GDP source and ligands capable of binding specifically to the full Galpha protein indicating that the molecule is a GPCR agonist;
   an increase in signal when using a GDP source and ligands capable of binding specifically to the full Galpha protein indicating that the molecule is a GPCR inverse agonist;
   an increase in signal when using a GTP source and ligands capable of binding specifically to the empty Galpha protein indicating that the molecule is a GPCR agonist;
   a decrease in signal when using a GTP source and ligands capable of binding specifically to the empty Galpha protein indicating that the molecule is a GPCR inverse agonist;
   a decrease in signal when using a GTP source and ligands capable of binding specifically to the full Galpha protein indicating that the molecule is a GPCR
   an increase in signal when using a GTP source and ligands capable of binding specifically to the full Galpha protein indicating that the molecule is a GPCR inverse agonist.

According to a second aspect, the invention relates to a method for determining the ability of a molecule to modulate the activation of a G-protein-coupled receptor (GPCR), said method comprising the following steps:

a) introduction, into a first container, of:
a membrane preparation carrying one or more GPCRs and one or more G proteins,
a GDP source or a GTP source,
a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
a GPCR agonist;

b) measurement of the RET signal emitted in the first container;

c) introduction into a second container of the same reagents as in step a) and of the test molecule;

d) measurement of the RET signal emitted in the second container obtained in step c);

e) comparison of the signals obtained in steps b) and d);
an increase in signal when using a GDP source and ligands capable of binding specifically to the empty Galpha protein indicating that the molecule is a GPCR agonist or positive allosteric modulator;
a decrease in signal when using a GDP source and ligands capable of binding specifically to the full Galpha protein indicating that the molecule is a GPCR agonist or positive allosteric modulator;
an increase in signal when using a GTP source and ligands capable of binding specifically to the empty Galpha protein indicating that the molecule is a GPCR agonist or positive allosteric modulator;
a decrease in signal when using a GTP source and ligands capable of binding specifically to the full Galpha protein indicating that the molecule is a GPCR agonist or positive allosteric modulator;
a decrease in signal when using a GDP source and ligands capable of binding specifically to the empty Galpha protein indicating that the molecule is a GPCR antagonist or negative allosteric modulator;
an increase in signal when using a GDP source and ligands capable of binding specifically to the full Galpha protein indicating that the molecule is a GPCR antagonist or negative allosteric modulator;
a decrease in signal when using a GTP source and ligands capable of binding specifically to the empty Galpha protein indicating that the molecule is a GPCR antagonist or negative allosteric modulator;
an increase in signal when using a GTP source and ligands capable of binding specifically to the full Galpha protein indicating that the molecule is a GPCR antagonist or negative allosteric modulator.

The description also relates to a method for measuring the activation of a G-protein-coupled receptor (GPCR), said method comprising the following steps:
a) introduction, into a first container, of:
   a membrane preparation carrying one or more GPCRs and one or more G proteins,
   a GDP source or a GTP source,
   a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
b) measurement of the RET signal emitted in the first container;
c) introduction of (i) in a second container, the same reagents as in step a) and a molecule capable of modulating GPCR activation or (ii) in the first container, a molecule capable of modulating GPCR activation;
d) measurement of the RET signal emitted in the second container or in the first container obtained in step c);
e) comparison of the signals obtained in steps b) and d), an increase or decrease in the signal obtained in step d) compared to that obtained in step b) indicating a modulation of GPCR activation.

The description also relates to a method for detecting the appearance or disappearance of an empty G protein or a full G protein in a G-protein-coupled receptor preparation (GPCR), said method comprising the following steps:
a) introduction, into a first container, of:
   a membrane preparation carrying one or more GPCRs and one or more Galpha proteins,
   a GDP source or a GTP source,
   a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
b) measurement of the RET signal emitted in the first container;
c) introduction of (i) in a second container, the same reagents as in step a) and a molecule capable of modulating GPCR activation or (ii) in the first container, a molecule capable of modulating GPCR activation;
d) measurement of the RET signal emitted in the second container or in the first container obtained in step c);
e) comparison of the signals obtained in steps b) and d), an increase or decrease in the signal obtained in step d) compared to that obtained in step b) indicating an appearance or disappearance of the empty G protein or the full G protein.

The description also relates to a method for determining the ability of a molecule to modulate the activation of a G protein-coupled receptor (GPCR), said method comprising the following steps:
a) introduction, into a first container, of:
   a membrane preparation carrying one or more GPCRs and one or more G proteins,
   a GDP source and a GTP source,
   a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
b) measurement of the RET signal emitted in the first container;
c) introduction (i) into a second container of the same reagents as in step a) and the test molecule or (ii) into the first container of the test molecule;
d) measurement of the RET signal emitted in the second container or in the first container obtained in step c);
e) comparison of the signals obtained in steps b) and d);
   an increase in signal when using a GDP source and ligands capable of binding specifically to the empty Galpha protein indicating that the molecule is a GPCR agonist;
   a decrease in signal when using a GDP source and ligands capable of binding specifically to the empty Galpha protein indicating that the molecule is a GPCR inverse agonist;
   a decrease in signal when using a GDP source and ligands capable of binding specifically to the full Galpha protein indicating that the molecule is a GPCR agonist;
   an increase in signal when using a GDP source and ligands capable of binding specifically to the full Galpha protein indicating that the molecule is a GPCR inverse agonist;
   an increase in signal when using a GTP source and ligands capable of binding specifically to the empty Galpha protein indicating that the molecule is a GPCR agonist;
   a decrease in signal when using a GTP source and ligands capable of binding specifically to the empty Galpha protein indicating that the molecule is a GPCR inverse agonist;
   a decrease in signal when using a GTP source and ligands capable of binding specifically to the full Galpha protein indicating that the molecule is a GPCR agonist;
   an increase in signal when using a GTP source and ligands capable of binding specifically to the full Galpha protein indicating that the molecule is a GPCR inverse agonist.

The description also relates to a method for determining the ability of a molecule to modulate the activation of a G protein-coupled receptor (GPCR), said method comprising the following steps:

a) introduction, into a first container, of:
a membrane preparation carrying one or more GPCRs and one or more G proteins,
a GDP source and a GTP source,
a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
a GPCR agonist;

b) measurement of the RET signal emitted in the first container;

c) introduction into a second container of the same reagents as in step a) and of the test molecule;

d) measurement of the RET signal emitted in the second container obtained in step c);

e) comparison of the signals obtained in steps b) and d);
an increase in signal when using a GDP source and ligands capable of binding specifically to the empty Galpha protein indicating that the molecule is a GPCR agonist or positive allosteric modulator;
a decrease in signal when using a GDP source and ligands capable of binding specifically to the full Galpha protein indicating that the molecule is a GPCR agonist or positive allosteric modulator;
an increase in signal when using a GTP source and ligands capable of binding specifically to the empty Galpha protein indicating that the molecule is a GPCR agonist or positive allosteric modulator;
a decrease in signal when using a GTP source and ligands capable of binding specifically to the full Galpha protein indicating that the molecule is a GPCR agonist or positive allosteric modulator.
a decrease in signal when using a GDP source and ligands capable of binding specifically to the empty Galpha protein indicating that the molecule is a GPCR antagonist or negative allosteric modulator;
an increase in signal when using a GDP source and ligands capable of binding specifically to the full Galpha protein indicating that the molecule is a GPCR antagonist or negative allosteric modulator;
a decrease in signal when using a GTP source and ligands capable of binding specifically to the empty Galpha protein indicating that the molecule is a GPCR antagonist or negative allosteric modulator;
an increase in signal when using a GTP source and ligands capable of binding specifically to the full Galpha protein indicating that the molecule is a GPCR antagonist or negative allosteric modulator.

Step a)

According to the first aspect of the present invention, step a) consists in introducing, in a first container, the following three elements:
a membrane preparation carrying one or more GPCRs and one or more G proteins,
a GDP source or a GTP source,
a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein.

In an embodiment of the first aspect, in step a), the first container does not contain the test molecule. It is used to measure the basal level of the RET signal and/or the background noise of the RET signal.

According to the second aspect of the present invention, step a) consists in introducing, in a first container, the following four elements:
a membrane preparation carrying one or more GPCRs and one or more G proteins,
a GDP source or a GTP source,
a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein,
a GPCR agonist.

In a preferred embodiment, the GTP source is a source of non-hydrolyzable or slowly hydrolyzable GTP, preferably selected from GTPgammaS (GTPγS), GppNHp and GppCp, preferably GTPgammaS (GTPγS).

In a preferred embodiment, the GDP source is GDP.

It must be understood that the method according to the invention does not provide for the introduction of both a GTP source and a GDP source.

The different elements can be introduced into the container sequentially in any order, or simultaneously or almost simultaneously. The mixing of the elements makes it possible to obtain a reaction solution adapted to the implementation of a RET. Other elements can be added to the container to adapt the solution to the implementation of the RET. For example, coelenterazine h (benzyl-coelenterazine) or bisdeoxycoelenterazine (DeepBlueC™) or didhydrocoelenterazine (coelenterazine-400a) or D-luciferin can be added.

Ligands are capable of binding specifically, either individually or in combination, to the empty Galpha protein or to the full Galpha protein. In a first embodiment, each of the two ligands can bind specifically and individually to the empty Galpha protein or to the full Galpha protein, i.e. one ligand can bind specifically to the empty Galpha protein or to the full Galpha protein in the presence and absence of the other ligand. In a second particular embodiment, the two ligands in combination can bind specifically to the empty Galpha protein or to the full Galpha protein, i.e. at least one of the two ligands can bind specifically to the empty Galpha protein or to the full Galpha protein only in the presence of the other ligand.

In a particular embodiment, a GDP source and ligands capable of binding specifically, either individually or in combination, to the empty Galpha protein are used. This embodiment makes it possible to distinguish the empty Galpha protein from the full Galpha protein bound to the GDP.

In another particular embodiment, a GDP source and ligands capable of binding specifically, either individually or in combination, to the full Galpha protein are used. This embodiment makes it possible to discriminate between the full Galpha protein bound to the GDP and the empty Galpha protein.

In another particular embodiment, a GTP source and ligands capable of binding specifically, either individually or in combination, to the empty Galpha protein are used. This embodiment makes it possible to distinguish between the empty Galpha protein and the full Galpha protein bound to GTP and/or to non-hydrolyzable/slowly hydrolyzable GTP. In another particular embodiment, a GTP source and ligands capable of binding specifically, either individually or in combination, to the full Galpha protein are used. This embodiment makes it possible to distinguish the full Galpha protein bound to GTP and/or to non-hydrolyzable/slowly hydrolyzable GTP from the empty Galpha protein.

Advantageously, the Galpha protein is selected from the Galphai1, Galphai2, Galphai3, Galphao1, Galphao2, Galphaq, Galpha12, Galpha13, Galphas, Galphaz, Galphat1, Galphat2, Galpha11, Galpha14, Galpha15, Galpha16 and Galphagus protein, preferably selected from the Galphai1, Galphai2 and Galphai3 protein.

In a particular embodiment, the first ligand is a peptide and the second ligand is an antibody or anybody fragment. For example, the first ligand is a peptide with the sequence Ser-Ser-Arg-Gly-Tyr-Tyr-His-Gly-Ile-Trp-Val-Gly-Glu-Glu-Gly-Arg-Leu-Ser-Arg (SEQ ID NO: 1) (peptide KB1753, which specifically recognizes the full Galpha protein bound to GTP) and the second ligand is an anti-Galphai antibody. For example, the anti-Galphai antibody is the antibody from the supplier Santa Cruz Biotechnologies product #SC13533 (clone R4, which recognizes the empty and full forms of the G protein).

In another particular embodiment, the first ligand and the second ligand are antibodies or antibody fragments.

In a particular embodiment, the antibody or antibodies used for the implementation of the process according to the invention is/are selected from DSV36S, DSV3S, DSV39S, DSV26S (DSV antibodies available from Cisbio Bioassays on request) and #SC13533.

In another particular embodiment, the first ligand is an sdAb of sequence selected from SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 and the second ligand is an anti-Galphai antibody, for example antibody #SC13533 or DSV36S antibody.

Of course, the first ligand and the second ligand must not compete when they bind to the Galpha protein, for example the first ligand and the second ligand do not bind the same epitope on the Galpha protein.

The skilled person will have no difficulty in adapting the concentration of GPCR agonist and the concentration of the test molecule, for example according to the chosen agonist, the test molecule and/or according to the desired characteristics.

Step b)

According to the three aspects of the present invention, step b) consists in measuring the RET signal emitted in the first container, i.e. the container obtained in step a). The measured signal corresponds to the signal obtained in the container in the absence of the test molecule. The measurement can be made by conventional methods widely known to the skilled person and does not pose any particular problem. A device is usually used to detect and measure the RET signal, such as the PHERAstar FS microplate reader (BMG Labtech) with TR-FRET or bioluminescence reading mode.

Step c)

According to the first aspect of the invention:
In an embodiment, step c) consists in introducing into a second container the same reagents as in step a) and the test molecule. Advantageously, the second container is prepared in the same way as the first container, the only difference being the presence in the second container of the test molecule. This embodiment is advantageous because it allows the measurement of the RET signal emitted in the first container and in the second container simultaneously. This embodiment also allows the simultaneous measurement of the RET signal emitted in one or more second containers. Thus, this embodiment is particularly advantageous since it allows to test several different molecules in parallel.
In another embodiment, step c) consists in introducing the test molecule into the first container. This embodiment has the advantage of using only one container for the implementation of the method according to the invention.

In a particular embodiment, the test molecule is introduced in increasing concentration to vary the amplitude of the decrease or increase in the signal.

According to the second aspect of the invention, step c) consists in introducing into a second container the same reagents as in step a) and the test molecule. Advantageously, the second container is prepared in the same way as the first container, the only difference being the presence in the second container of the test molecule. Thus, the measurement of the RET signal emitted in the first container and in the second container is performed simultaneously. This also allows the simultaneous measurement of the RET signal emitted in one or more second containers. This allows several different molecules to be tested in parallel.

In a particular embodiment, the test molecule or GPCR agonist is introduced in increasing concentration to vary the amplitude of the signal decrease or increase.

Step d)

According to the three aspects of the present invention, step d) consists in measuring the RET signal emitted in the second container or in the first container obtained in step c). The measured signal corresponds to the signal obtained in the container in the presence of the test molecule (±an agonist for the second aspect). As in step b), the measurement can be done by conventional methods widely known to the skilled person and does not pose any particular problem. A device is usually used to detect and measure the RET signal, such as the PHERAstar FS microplate reader (BMG Labtech) with TR-FRET or bioluminescence reading mode.

Step e)

According to the three aspects of the present invention, step e) consists in comparing the signals obtained in steps b) and d).

The skilled person can easily compare the signals in steps b) and d) and define a threshold allowing him to qualify the increase or decrease, for example a difference between the signals of more than 5%, more than 10%, more than 15%, more than 20% or more than 25%. For example, the ratio between the signals in steps b) and d) can be calculated. In general, for a given pair of ligands, the larger the difference between the signals, the greater the ratio between the signals will be and the greater the modulation of GPCR activation (e.g. activation or inhibition). However, the difference between the signals may vary according to the ligand pair used for the implementation of the process according to the invention. The level of modulation of GPCR activation makes it possible to identify more or less agonist, antagonist, reverse agonist, positive allosteric modulator or negative allosteric modulator molecules.

According to the first aspect:
an increase in signal when using a GDP source and ligands capable of binding specifically to the empty Galpha protein indicating that the molecule is a GPCR agonist;
a decrease in signal when using a GDP source and ligands capable of binding specifically to the empty Galpha protein indicating that the molecule is a GPCR inverse agonist;
a decrease in signal when using a GDP source and ligands capable of binding specifically to the full Galpha protein indicating that the molecule is a GPCR agonist;
an increase in signal when using a GDP source and ligands capable of binding specifically to the full Galpha protein indicating that the molecule is a GPCR inverse agonist;
an increase in signal when using a GTP source and ligands capable of binding specifically to the empty Galpha protein indicating that the molecule is a GPCR agonist;
a decrease in signal when using a GTP source and ligands capable of binding specifically to the empty Galpha protein indicating that the molecule is a GPCR inverse agonist;
a decrease in signal when using a GTP source and ligands capable of binding specifically to the full Galpha protein indicating that the molecule is a GPCR agonist;
an increase in signal when using a GTP source and ligands capable of binding specifically to the full Galpha protein indicating that the molecule is a GPCR inverse agonist.

According to the second aspect:
an increase in signal when using a GDP source and ligands capable of binding specifically to the empty Galpha protein indicating that the molecule is a GPCR agonist or positive allosteric modulator;
a decrease in signal when using a GDP source and ligands capable of binding specifically to the full Galpha protein indicating that the molecule is a GPCR agonist or positive allosteric modulator;
an increase in signal when using a GTP source and ligands capable of binding specifically to the empty Galpha protein indicating that the molecule is a GPCR agonist or positive allosteric modulator;
a decrease in signal when using a GTP source and ligands capable of binding specifically to the full Galpha protein indicating that the molecule is a GPCR agonist or positive allosteric modulator.
a decrease in signal when using a GDP source and ligands capable of binding specifically to the empty Galpha protein indicating that the molecule is a GPCR antagonist or negative allosteric modulator;
an increase in signal when using a GDP source and ligands capable of binding specifically to the full Galpha protein indicating that the molecule is a GPCR antagonist or negative allosteric modulator;
a decrease in signal when using a GTP source and ligands capable of binding specifically to the empty Galpha protein indicating that the molecule is a GPCR antagonist or negative allosteric modulator;
an increase in signal when using a GTP source and ligands capable of binding specifically to the full Galpha protein indicating that the molecule is a GPCR antagonist or negative allosteric modulator.

Labeling of the Ligand with a Member of a RET Partner Pair

A ligand can be labeled directly or indirectly.

Direct labeling of the ligand with a member of a RET partner pair, for example a fluorescent compound when using FRET, can be carried out by conventional methods known to the skilled person, based on the presence of reactive groups on the ligand. For example, when the ligand is an antibody or antibody fragment, the following reactive groups may be used: the terminal amino group, carboxylate groups of aspartic and glutamic acids, amino groups of lysines, guanidine groups of arginines, thiol groups of cysteines, phenol groups of tyrosines, indole rings of tryptophans, thioether groups of methionines, imidazole groups of histidines.

Reactive groups can form a covalent bond with a reactive group carried by a member of a

RET partner pair. The appropriate reactive groups, carried by the member of a RET partner pair, are well known to the skilled person, e.g. a donor compound or an acceptor compound functionalized with a maleimide group will for example be capable of covalently binding with thiol groups carried by cysteines carried by a protein or peptide, e.g. an antibody or antibody fragment. Similarly, a donor/acceptor compound carrying an N-hydroxysuccinimide ester will be able to covalently bind to an amine containing a protein or peptide.

The ligand may also be labeled with a fluorescent or bioluminescent compound indirectly, for example by introducing into the measurement medium an antibody or antibody fragment, which is itself covalently bound to an acceptor/donor compound, this second antibody or antibody fragment specifically recognizing the ligand.

Another very traditional indirect labeling method consists in attaching biotin to the ligand to be labeled, then incubating this biotinylated ligand in the presence of streptavidin labeled with an acceptor/donor compound. Appropriate biotinylated ligands can be prepared by techniques well known to the skilled person; for example, Cisbio Bioassays markets fluorophore-labeled streptavidin under the trade name “d2” (item 610SADLA).

In the context of the invention, the first ligand and the second ligand are each labeled with a member of a RET partner pair, one of the members of the pair being a fluorescent donor or luminescent donor compound and the other member of the pair being a fluorescent acceptor compound or a non-fluorescent acceptor compound (quencher).

Labeling for the Implementation of FRET

In a particular embodiment, the first ligand and the second ligand are each labeled with a member of a FRET partner pair, i.e. a fluorescent donor compound or a fluorescent energy-accepting compound.

The selection of the FRET partner pair to obtain a FRET signal is within the reach of the skilled person. For example, donor-acceptor pairs that can be used to study FRET phenomena are described in the work by Joseph R. Lakowicz (Principles of fluorescence spectroscopy, 2nd edition 338), to which the skilled person may refer.

Fluorescent Donor Compounds

Long-lived energy-donating fluorescent compounds (>0.1 ms, preferably in the range 0.5 to 6 ms), in particular rare earth chelates or cryptates, are advantageous since they allow time-resolved FRET without having to deal with a large part of the background noise emitted by the measurement medium. For this reason, they are generally preferred for the implementation of the process according to the invention. Advantageously, these compounds are lanthanide complexes. These complexes (such as chelates or cryptates) are particularly suitable as a member of the energy-donating FRET pair.

Complexes of europium (Eu3+), terbium (Tb3+), dysprosium (Dy3+), samarium (Sm3+), neodymium (Nd3+), ytterbium (Yb3+) or erbium (Er3+) are rare earth complexes also suitable for the invention, with europium (Eu3+) and terbium (Tb3+) complexes being particularly preferred.

A large number of rare earth complexes have been described and several are currently being commercialized by Perkin Elmer, Invitrogen and Cisbio Bioassays.

Examples of rare earth chelates or cryptates suitable for the purpose of the invention are:
Lanthanide cryptates, containing one or more pyridine units. Such rare earth cryptates are described, for example, in patents EP 0 180 492, EP 0 321 353, EP 0 601 113 and in international application WO 01/96 877. The cryptates of terbium (Tb3+) and europium (Eu3+) are particularly suitable for the purposes of the present invention. Lanthanide cryptates are marketed by Cisbio Bioassays. Examples of this are, but are not limited to, europium cryptates of the formulas below (which can be coupled to the compound to be labeled via a reactive group, here for example an NH₂ group):

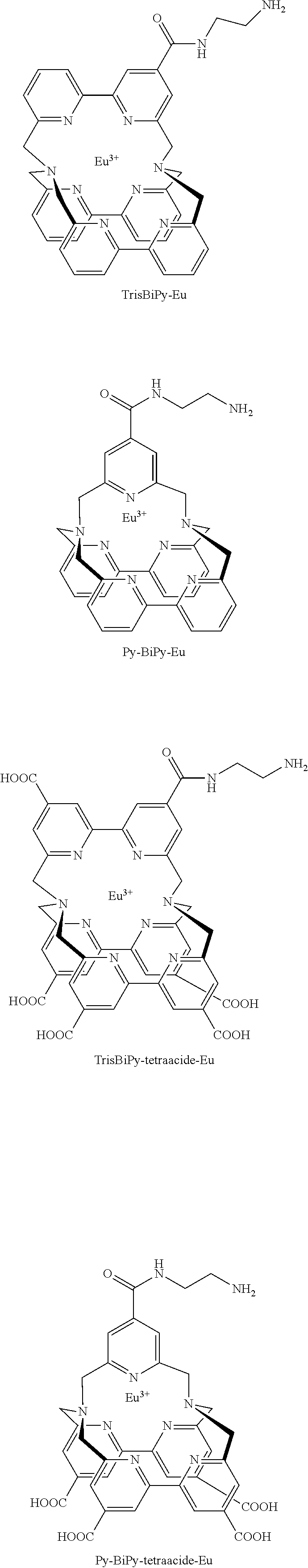

Lanthanide chelates described in particular in patents U.S. Pat. Nos. 4,761,481, 5,032,677, 5,055,578, 5,106,957, 5,116,989, 4,761,481, 4,801,722, 4,794,191, 4,637,988, 4,670,572, 4,837,169, 4,859,777. Patents EP 0 403 593, U.S. Pat. Nos. 5,324,825, 5,202,423, 5,316,909 describe chelates composed of a nonadentate ligand such as terpyridine. Lanthanide chelates are marketed by Perkin Elmer.
Lanthanide complexes consisting of a chelating agent, such as tetraazacyclododecane, substituted by a chromophore having aromatic rings, such as those described by Poole R. et al. in Biomol. Chem, 2005, 3, 1013-1024 “Synthesis and characterisation of highly emissive and kinetically stable lanthanide complexes suitable for usage in cellulo”, can also be used. The complexes described in application WO 2009/10580 can also be used.
The lanthanide cryptates described in patents EP 1 154 991 and EP 1 154 990 are also available.
   the terbium cryptate of the formula below (which can be coupled to a compound to be labeled via a reactive group, here for example an NH₂ group):

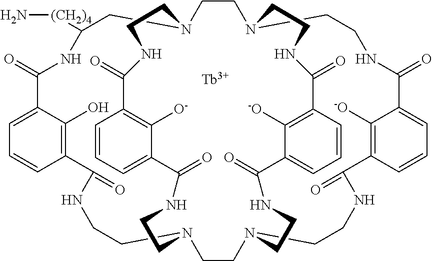

and the synthesis of which is described in international application WO 2008/063721 (compound 6a page 89).
The terbium cryptate Lumi4-Tb from Lumiphore, marketed by Cisbio Bioassays.
The quantum dye from Research Organics, of the formula below (which can be coupled to the compound to be labeled via a reactive group, here NCS):

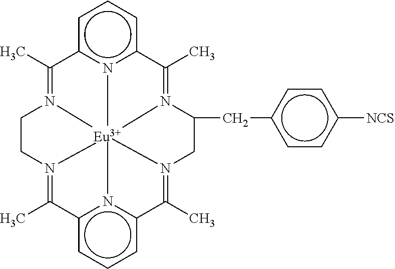

Ruthenium chelates, in particular complexes consisting of a ruthenium ion and several bipyridines such as ruthenium(II) tris(2,2′-bipyridine).
Terbium chelate DTPA-cs124 Tb, marketed by Life Technologies of the formula below (which can be coupled to the compound to be labeled via a reactive group R) and whose synthesis is described in patent U.S. Pat. No. 5,622,821.

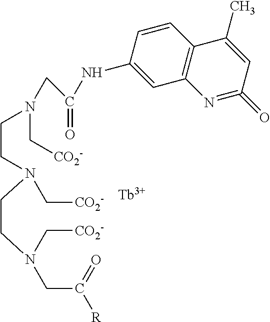

Terbium chelate of the formula below and described by Latva et al. (Journal of Luminescence 1997, 75(2): 149-169):

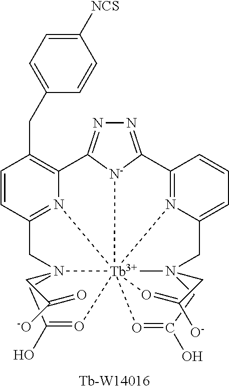

Advantageously, the fluorescent donor compound is a FRET partner selected from: europium cryptate, europium chelate, terbium chelate, terbium cryptate, ruthenium chelate, quantum dot, allophycocyanins, rhodamines, cyanins, squarains, coumarins, proflavins, acridines, fluoresceins, boron-dipyrromethene derivatives and nitrobenzoxadiazole.

Particularly advantageously, the fluorescent donor compound is a FRET partner selected from: europium cryptate; europium chelate; terbium chelate; terbium cryptate; ruthenium chelate; and quantum dot; europium and terbium chelates and cryptates being particularly preferred.

Fluorescent Acceptor Compounds

Fluorescent acceptor compounds can be selected from the following group: allophycocyanins, in particular those known under the trade name XL665; luminescent organic molecules, such as rhodamines, cyanins (such as Cy5), squarains, coumarins, proflavins, acridins, fluoresceins, boron-dipyrromethene derivatives (marketed as “Bodipy”), fluorophores known as “Atto”, fluorophores known as “DY”, compounds known as “Alexa”, nitrobenzoxadiazole. Advantageously, fluorescent acceptor compounds are selected from al lophycocyanins, rhodamines, cyanins, squarains, coumarins, proflavins, acridins, fluoresceins, boron-dipyrromethene derivatives, nitrobenzoxadiazole.

The terms “cyanins” and “rhodamines” should be understood as “cyanine derivatives” and “rhodamine derivatives” respectively. The skilled person is familiar with these different fluorophores, which are available on the market.

“Alexa” compounds are marketed by Invitrogen; “Atto” compounds are marketed by Attotec; “DY” compounds are marketed by Dyomics; “Cy” compounds are marketed by Amersham Biosciences; other compounds are marketed by various chemical reagent suppliers, such as Sigma, Aldrich or Acros.

The following fluorescent proteins can also be used as fluorescent acceptor compounds: cya fluorescent proteins (AmCyan1, Midori-Ishi Cyan, mTFP1), green fluorescent proteins (EGFP, AcGFP, TurboGFP, Emerald, Azami Green, ZsGreen), yellow fluorescent proteins (EYFP, Topaz, Venus, mCitrine, YPet, PhiYFP, ZsYellow1, mBanana), orange and red fluorescent proteins (Orange kusibari, mOrange, tdtomato, DsRed, DsRed2, DsRed-Express, DsRed-Monomer, mTangerine, AsRed2, mRFP1, JRed, mCherry, mStrawberry, HcRed1, mRaspberry, HcRed-Tandem, mPlim, AQ143), fluorescent proteins in far red (mKate, mKate2, tdKatushka2).

Advantageously, the fluorescent acceptor compound is a FRET partner selected from: allophycocyanins, rhodamines, cyanins, squarains, coumarins, proflavins, acridins, fluoresceins, boron-dipyrromethene derivatives, nitrobenzoxadiazole and a quantum dot, GFP, GFP variants selected from GFP10, GFP2 and eGFP, YFP, YFP variants selected from eYFP, YFP topaz, YFP citrine, YFP venus and YPet, mOrange, DsRed.

Labeling for the Implementation of BRET

In a particular embodiment, the first ligand and the second ligand are each labeled with a member of a BRET partner pair, i.e. a luminescent donor compound or a fluorescent energy-accepting compound.

A ligand can be labeled directly or indirectly.

The direct labeling of the ligand by a luminescent donor compound or a protein-type fluorescent acceptor compound, member of a BRET partner pair, can be carried out by the classical methods known to the skilled person and in particular described in the article by Tank Issad and Ralf Jockers (Bioluminescence Resonance Energy Transfer to Monitor Protein-Protein Interactions, Transmembrane Signaling Protocols pp 195-209, Part of the Methods in Molecular BiologyTM book series MIMB, volume 332) to which the skilled person may refer.

The direct labeling of the ligand by an organic molecule-type fluorescent acceptor compound, member of a BRET partner pair, can be carried out by the classical methods known to the skilled person, based on the presence of reactive groups on the ligand as mentioned above. For example, when the ligand is an antibody or antibody fragment, the following reactive groups may be used: the terminal amino group, carboxylate groups of aspartic and glutamic acids, amino groups of lysines, guanidine groups of arginines, thiol groups of cysteines, phenol groups of tyrosines, indole rings of tryptophans, thioether groups of methionines, imidazole groups of histidines.

Reactive groups can form a covalent bond with a reactive group carried by a member of a BRET partner pair. The appropriate reactive groups, carried by the member of a BRET partner pair, are well known to the skilled person, for example an acceptor compound functionalized with a maleimide group will be able to bind covalently with thiol groups carried by cysteines carried by a protein or peptide, for example an antibody or antibody fragment. Similarly, an acceptor compound carrying an N-hydroxysuccinimide ester will be capable of covalently binding to an amine containing a protein or peptide.

The ligand may also be labeled with a bioluminescent or fluorescent compound indirectly, for example by introducing into the measurement medium an antibody or antibody fragment, which is itself covalently bound to an acceptor/donor compound, this second antibody or antibody fragment specifically recognizing the ligand.

Another very traditional indirect labeling method consists in attaching biotin to the ligand to be labeled, then incubating this biotinylated ligand in the presence of streptavidin labeled with an acceptor/donor compound. Appropriate biotinylated ligands can be prepared by techniques well known to the skilled person; for example, Cisbio Bioassays markets fluorophore-labeled streptavidin under the trade name “d2” (item 610SADLA).

The selection of the BRET partner pair to obtain a BRET signal is within the reach of the skilled person. For example, donor-acceptor pairs that can be used to study BRET phenomena are described in particular in the article by Dasiel O. Borroto-Escuela (BIOLUMINESCENCE RESONANCE ENERGY TRANSFER (BRET) METHODS TO STUDY G PROTEIN-COUPLED RECEPTOR-RECEPTOR TYROSINE KINASE HETERORECEPTOR COMPLEXES, Cell Biol. 2013; 117: 141-164), which the skilled person may refer.

Luminescent Donor Compounds

In a particular embodiment, the luminescent donor compound is a BRET partner selected from: Luciferase (luc), Renilla Luciferase (Rluc), variants of Renilla Luciferase (Rluc8) and Firefly Luciferase.

Fluorescent Acceptor Compounds

In a particular embodiment, the fluorescent acceptor compound is a BRET partner selected from: allophycocyanins, rhodamines, cyanins, squarains, coumarins, proflavins, acridins, fluoresceins, boron-dipyrromethene derivatives, nitrobenzoxadiazole, a quantum dot, GFP, GFP variants (GFP10, GFP2, eGFP), YFP, YFP variants (eYFP, YFP topaz, YFP citrine, YFP venus, YPet), mOrange, DsRed.

Kit

The invention also relates to a kit for implementing the method according to the invention, comprising:
a first ligand of the G protein alpha subunit (Galpha protein) labeled with a first member of a RET partner pair and a second ligand of the Galpha protein labeled with a second member of the RET partner pair, said ligands being capable of binding specifically, either individually or in combination, to the empty Galpha protein or to the full Galpha protein; and
a set of instructions.

In a particular embodiment, the kit further comprises a GDP source or a GTP source.

In a particular embodiment, the kit further comprises a Galpha protein and/or a membrane preparation carrying one or more GPCRs and one or more G proteins.

The kit may also comprise dilution buffer(s) for the reagents.

### EXAMPLES

Protocol for Obtaining Anti-Galphai1 Protein Antibodies

The recombinant TST-Galphai1 protein (Galphai1 protein of UniProt sequence P63096-1 N-terminally tagged with the TwinStreptag (TST) tag (IBA) via a TEV linker) was produced in Sf9 insect cells (infection with a baculovirus encoding said protein) then purified on an affinity column via the TwinStreptag (TST) tag (Strep-Tactin Superflow high capacity resin (IBA, Catalogue: 2-1208-002)).

BALB/c mice were immunized by injection of the TST-Galphai1 protein previously diluted in buffer containing GTPgS (HEPES 20 mM pH8, NaCl 100 mM, MgCl2 3 mM, CHAPS 11 mM, GTPgS 100 μM). The first injection was followed by three boosters at one-month intervals. Fifteen days after each injection, blood punctures were performed on the mice to check for the presence of an immune response.

For this purpose, an ELISA test was set up. The TST-Galphai1 protein previously diluted at 20 μg/mL in buffer containing GTPgS (Tris HCl 20 mM pH8.5, NaCl 140 mM, EDTA 2 mM, MgCl2 10 mM, BSA 0.1%, GTPgS 1 μM) was adsorbed via the TwinStreptag tag on 96-well plates containing Strep-Tactin®XT (IBA, Catalogue: 2-4101-001). For this purpose, 100 μl of protein was added in each well and incubated for 2 h at 37° C. followed by three washes in buffer PBS 1×, 0.05% Tween20.

Serial dilutions of a factor of 10 to 100 million of the blood punctures were then added at 100 μL/well and incubated for 2 h at 37° C. Non-protein bound antibodies were removed by three washing steps in 1× PBS, 0.05% Tween20 buffer and then detection of the bound antibodies was performed using a secondary mouse anti-Fc antibody bound to HRP (horseradish peroxidase) (Sigma #A0168 diluted 1/10,000 in PBS, BSA 0.1%). After 1 hour of incubation at 37° C. and then three washes in 1× PBS, 0.05% Tween20 buffer, the HRP was determined by colorimetric assay at 450 nm following the incubation of its TMB substrate (3,3′,5,5,5′-Tetramethylbenzidine, Sigma #T0440) for 20 min at room temperature under stirring.

In order to ensure that the antibodies detected by the ELISA test were directed against the Galphail protein and not against the TwinStrepTag, the same punctures were tested on the ELISA test after pre-incubation with an excess of another orthogonal protein labeled with the TwinStrepTag (SNAPTag-TwinStrpeTag). Thus, anti tag antibodies bind to the orthogonal labeled protein and therefore not to the Galphai1 protein attached to the bottom of the wells; in which case no HRP signal or a decrease in the HRP signal is detected.

Mice with the best antibody titers and the lowest signal drop in the anti tag control case were selected for the next step of lymphocyte hybridization, also called fusion. The spleen of the mice was recovered and a mixture of lymphocytes and plasmocysts from this spleen was fused in vitro with a myeloma cell line in the presence of a polyethylene glycol type cell fusion catalyst. A mutant myeloma cell line, missing the enzyme hypoxanthine-guanine phosphoribosyltransferase (HGPRT), was used to allow selection of hybrid cells, called hybridomas. These cells were cultured in a medium containing hypoxanthine, aminopterin (methotrexate) and thymidine (HAT medium) to allow the elimination of unfused myeloma cells and thus select hybridomas of interest. Unfused spleen cells die because they are unable to proliferate in vitro. Thus, only hybridomas survived.

These hybridomas were then grown in culture plates. The supernatants of these hybridomas were then tested to assess their ability to produce anti-Galphai1 protein antibodies. For this purpose, an ELISA test as described above was carried out.

In order to evaluate the selectivity of antibodies between the different forms of the Galphai1 protein (GDP-bound full form vs. GTPgS-bound full form vs. empty form), the test was performed in parallel on TST-Galphai1 protein conditions pre-incubated in the buffer containing either GDP at 1 μM, GTPgS at 1 μM or no nucleotide. The best hybridomas were then cloned with a limit dilution step to obtain hybridoma clones.

The hybridoma clones of interest were then injected into mice (intraperitoneal injection) to allow the production of antibodies in large quantities in the ascites fluid.

The antibodies were then purified by affinity chromatography on columns with resins containing protein A.

The monoclonal antibodies thus obtained were labeled with fluorescent probes, for example, as explained above, for use in the method of the invention.

Materials
the cell membranes expressing the receptors studied and the Galphai protein were purchased from Perkin Elmer. The table below lists the cell lines and item numbers of the different samples used:

| Receptor | Cell line | Supplier | Item number |
|---|---|---|---|
| Delta Opioid | HEK293 | Perkin Elmer | 6110549400UA |
| Delta Opioid | CHO-K1 | Perkin Elmer | RBHODM400UA |
| Galanin R1 (GALR1) | HEK293 | Perkin Elmer | 6110537400UA |
| NOP (ORL-1) | HEK293 | Perkin Elmer | RBHORLM400UA |
| 5HT1A | HEK293- | Perkin Elmer | RBHS1AM400UA |
| | EBNA | | |

the anti-Galphai antibody SC13533 was purchased from Santa Cruz Biotechnology (catalogue number SC13533). This antibody does not specifically bind the three forms of the Galpha protein (empty Galpha protein, GDP-bound full Galpha protein and full Galpha protein bound to GTP or non-hydrolyzable or slowly hydrolyzable GTP).
DSV36S, DSV3S, DSV39S, DSV26S antibodies have been generated by Cisbio Bioassays and are available from Cisbio Bioassays on request (under the respective product numbers DSV36S, DSV3S, DSV39S, DSV26S). The antibodies were labeled with compatible fluorescent probes for TR-FRET detection (red—d2 acceptor or Lumi4Tb donor). The biotinylated peptide KB1753 was generated by Cisbio Bioassays.
   the DSV36S antibody specifically binds the form of the Galpha GTP or GTP protein that is non-hydrolyzable or slowly hydrolyzable relative to the forms of the empty Galpha protein and GDP
   the DSV3S antibody specifically binds the form of the empty Galpha protein to the GDP and GTP or GTP forms that are non-hydrolyzable or slowly hydrolyzable.
   the DSV26S antibody specifically binds the form of the empty Galpha protein to the GDP and GTP or GTP forms that are non-hydrolyzable or slowly hydrolyzable.
   the DSV39S antibody does not specifically bind the three forms of the Galpha protein (empty Galpha protein, full Galpha protein bound to GDP and full Galpha protein bound to GTP or to GTP that is not hydrolyzable or slowly hydrolyzable).
   the peptide KB1753 specifically binds the form of the Galpha GTP or GTP protein that is non-hydrolyzable or slowly hydrolyzable relative to the forms of the empty Galpha protein and GDP.
   the ligand pair peptide KB1753/anti-Galphai antibody SC13533 specifically binds the form of the Galpha GTP or GTP protein that is non-hydrolyzable or slowly hydrolyzable relative to the forms of the empty Galpha protein and GDP.
   the ligand pair anti-Galphai antibody DSV36S/anti-Galphai antibody SC13533 specifically binds the form of the Galpha GTP or GTP protein that is non-hydrolyzable or slowly hydrolyzable relative to the forms of the empty Galpha protein and GDP.
   the ligand pair anti-Galphai antibody DSV39S/anti-Galphai antibody SC13533 specifically binds the form of the Galpha GTP or GTP protein that is non-hydrolyzable or slowly hydrolyzable relative to the forms of the empty Galpha protein and GDP.
   the ligand pair anti-Galphai DSV3S antibody/anti-Galphai DSV36S antibody specifically binds the form of the Galpha GTP or GTP protein that is non-hydrolyzable or slowly hydrolyzable relative to the forms of the empty Galpha protein and GDP.
   the ligand pair anti-Galphai antibody DSV39S/anti-Galphai antibody DSV26S specifically binds the form of the empty Galpha protein relative to the forms of the Galpha GDP and GTP or non-hydrolyzable or slowly hydrolyzable GTP protein.
streptavidin labeled with the red acceptor XL665 was generated by Cisbio Bioassays (catalogue number 610SAXLB).
GTP, GDP and GTPγS nucleotides were purchased from Sigma Aldrich (catalogue numbers G8877, G7127 and G8634 respectively).
the agonist of GPCR Delta Opioid SNC162 and the antagonist of GPCR Delta Opioid Naltrindole were purchased from Tocris (catalogue numbers 1529 and 0740 respectively).
384-well low-volume, white, white-backed plates were purchased from Greiner Bio One (Catalogue number 784075).

Method

Preparation of Reagents:

All reagents were diluted in TrisHCl 50 mM pH 7.4, MgCl2 10 mM, NaCl 10 mM, BSA 0.1% buffer. The membranes were prepared 4× to distribute 1 μg/well (except for other specifications). The GDP, GTP or GTPγS nucleotides and test compounds (agonists or antagonists) were pre-mixed and prepared 4× to obtain the final concentrations in the wells mentioned in the graphs. All anti-Galphai reagents used for detection were prepared 4× to target the following final concentrations in the following wells: sdAbs-d2 (50 nM); antibody SC13533-d2 (0.1 nM); antibody SC13533-Lumi4Tb (0.25 nM); antibody DSV36S-d2 (0.1 nM); antibody DSV36S-Lumi4Tb (0.25 nM); antibody DSV3S-d2 (10 nM); antibody DSV39S-d2 (10 nM); antibody DSV26S-Lumi4Tb (0.25 nM); peptide KB1753-Biotin (50 nM) in combination with streptavidin-XL665 (SA-XL) (25 nM) (pre-incubated 30 minutes at room temperature before distribution in plates).
Distribution of reagents in the 384-well plates:
   Membranes expressing GPCR and G protein: 5 μL
   Nucleotides (GDP or GTP or GTPγS) ±Test compounds (agonists and/or antagonists): 5 μL
   Anti-Galphai-acceptor ligand (d2 or XL665): 5 μL
   Anti-Galphai-donor ligand (Lumi4Tb): 5 μL

The non-specific signal (background fluorescence noise) was measured with wells containing only the two detection reagents (labeled with the donor and acceptor), the other components having been replaced by their dilution buffer.

Reading the HTRF Signal:

The plates were incubated at 21° C. for 3 h or 20 h and then the HTRF signal was measured on the PHERAstar reader (BMG Labtech) with the following configuration:
Module: HTRF (Excitation 337nm, Emission 665nm and 620nm)
Excitation: laser, 40 flashes
Reading window: delays: 60 μs—Integration: 400 μs

Signal Processing:

From the raw signals at 665 nm and 620 nm, the HTRF Ratio was calculated according to the following formula:

HTRF ratio=Signal at 665 nm/Signal at 620 nm*10,000

Test Formats

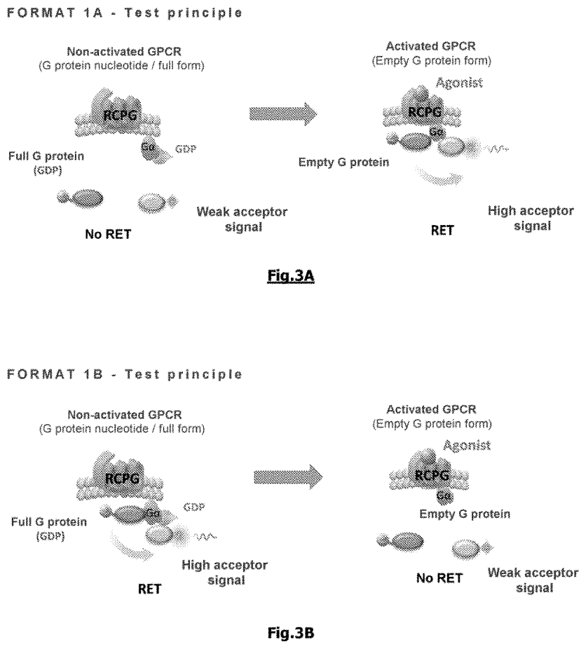

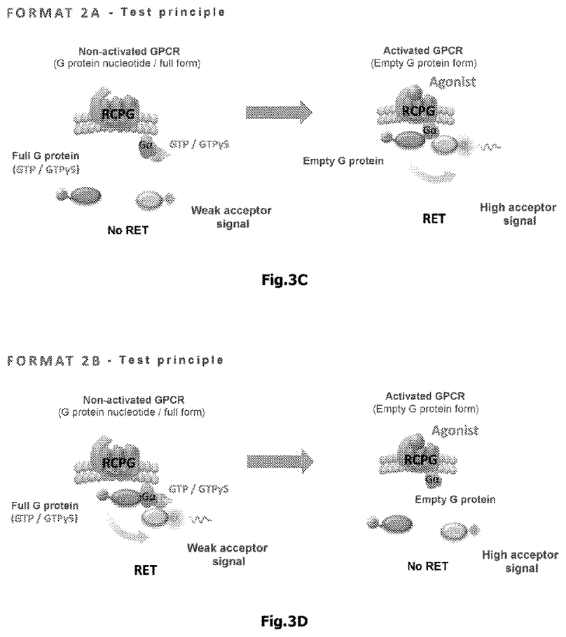

FIG. 3A illustrates the discrimination between the empty Galpha protein form and the GDP-bound full Galpha protein form (format 1A). After activation of GPCR and Galpha protein by a GPCR agonist, the increase in the proportion of the empty Galpha protein form is specifically detected using two ligands labeled with RET partners. Activation of GPCR results in an increase in the RET signal.

FIG. 3B illustrates the discrimination of the GDP-bound full Galpha protein form from the empty Galpha protein form (format 1B). After activation of GPCR and Galpha protein by a GPCR agonist, the decrease in the proportion of the GDP-bound full Galpha protein form (due to an increase in the proportion of the empty Galpha protein) is detected specifically using two ligands labeled with RET partners. Activation of GPCR results in a decrease in the RET signal.

FIG. 3C illustrates the discrimination of the empty Galpha protein form from the full

Galpha protein form bound to GTP or bound to GTPγS (format 2A). After activation of GPCR and Galpha protein by a GPCR agonist, the increase in the proportion of the empty Galpha protein form is specifically detected using two ligands labeled with RET partners. Activation of GPCR results in an increase in the RET signal.

FIG. 3D illustrates the discrimination of the full Galpha protein form bound to GTP or bound to GTPyS from the empty Galpha protein form (format 2B). After activation of GPCR and Galpha protein by a GPCR agonist, the decrease in the proportion of the full Galpha protein form bound to GTP or bound to GTPγS (due to an increase in the proportion of the empty Galpha protein form) is specifically detected using two ligands labeled with RET partners. Activation of GPCR results in a decrease in the RET signal.

### EXAMPLES 1 TO 4

### Activation Test According to Format 2B: Discrimination of the GTP or GTPγS form from the Empty form with Detection Ligand Pairs Labeled with TR-FRET partners

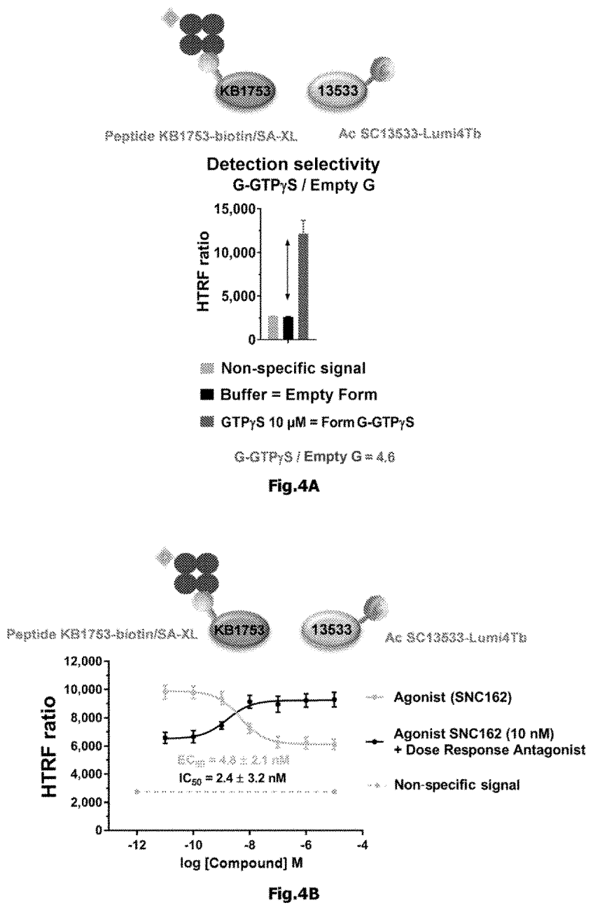

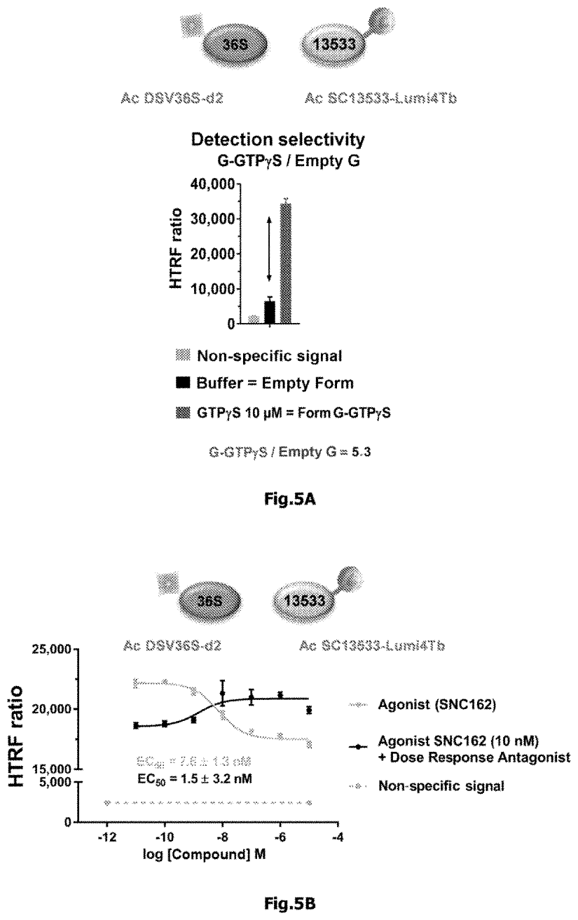

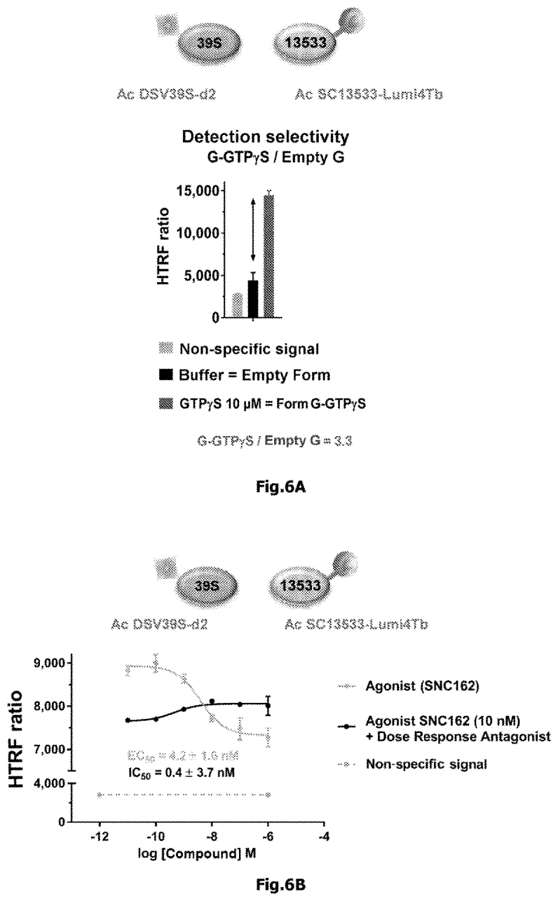

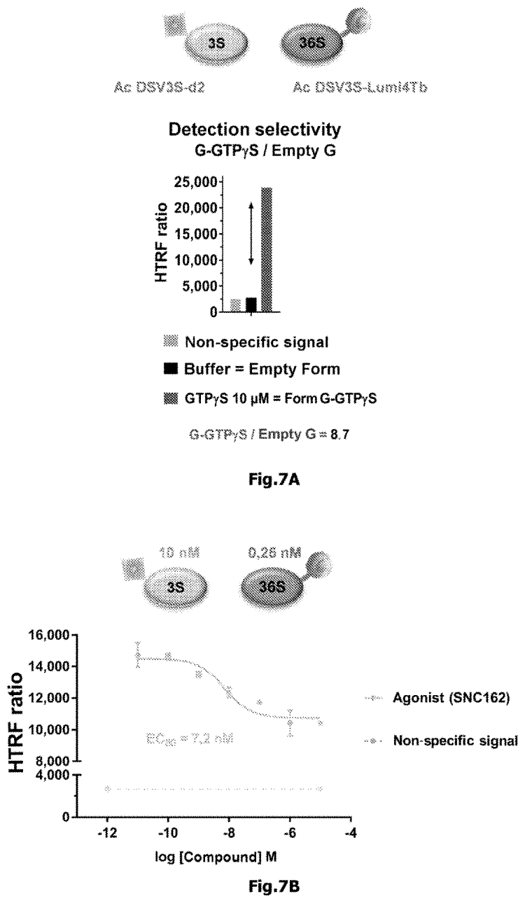

First, the ability of detection ligand pairs to specifically detect full Galpha protein bound to GTP or bound to GTPγS against empty Galpha protein was demonstrated using HEK293 cell membranes expressing the Delta Opioid GPCR and Galphai protein. The membranes were incubated with an excess of GTPγS (10 μM), allowing the G protein to be loaded with the nucleotide (full Galpha protein form bound to GTPγS), or with buffer alone (empty Galpha protein form). The difference in TR-FRET signal (HTRF ratio) observed between these two conditions shows that the 4 detection ligand pairs used (Peptide KB1753-biotin/SA-XL/anti-Galphai antibody SC13533-Lumi4Tb—Example 1/FIG. 4A; anti-Galphai antibody DSV36S-d2/anti-Galphai antibody SC13533-Lumi4Tb—Example 2/FIG. 5A; anti-Galphai antibody DSV39S-d2/anti-Galphai antibody SC13533-Lumi4Tb—example 3/FIG. 6A; anti-Galphai antibody DSV3S-d2/anti-Galphai antibody DSV36S-Lumi4Tb—example 4/FIG. 7A) can discriminate between the Galpha protein form bound to GTPγS and the empty Galpha protein form.

In a second step, the ability of a GPCR agonist to modulate the proportion of Galpha protein in empty form was tested with the same membranes and detection ligand pairs. HEK293 cell membranes expressing Delta Opioid GPCR and Galphai protein were incubated with GTPγS (10 nM) and GPCR agonist (SNC162) in increasing concentration. The decrease in the TR-FRET signal (HTRF ratio) generated by stimulation with the agonist means that the proportion of empty Galpha protein form increases. Thus, the GPCR receptor activated by its agonist causes the GTPgS to exit the G protein which then passes into empty form and causes the TR-FRET signal to decrease. In a second condition, activation with a fixed concentration of GPCR agonist SNC162 (10 nM) was inhibited by an increasing concentration of GPCR antagonist (Naltrindole). This activation inhibition is observed by the increase in the TR-FRET signal (HTRF ratio). These results are shown in FIGS. 4B (Example 1), 5B (Example 2), 6B (Example 3) and 7B (Example 4, in which the second condition was not implemented).

### EXAMPLE 5

### Activation Test According to Format 1A: Discrimination of the Empty Galpha Protein Form of the Full Galpha Protein Form Bound to GDP) with a Detection Ligand Pair Labeled with TR-FRET Partners

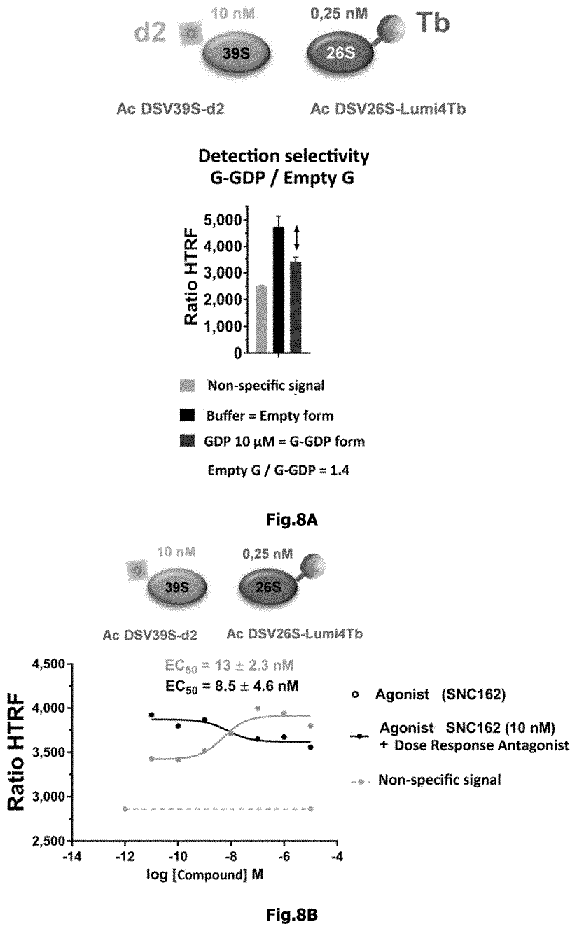

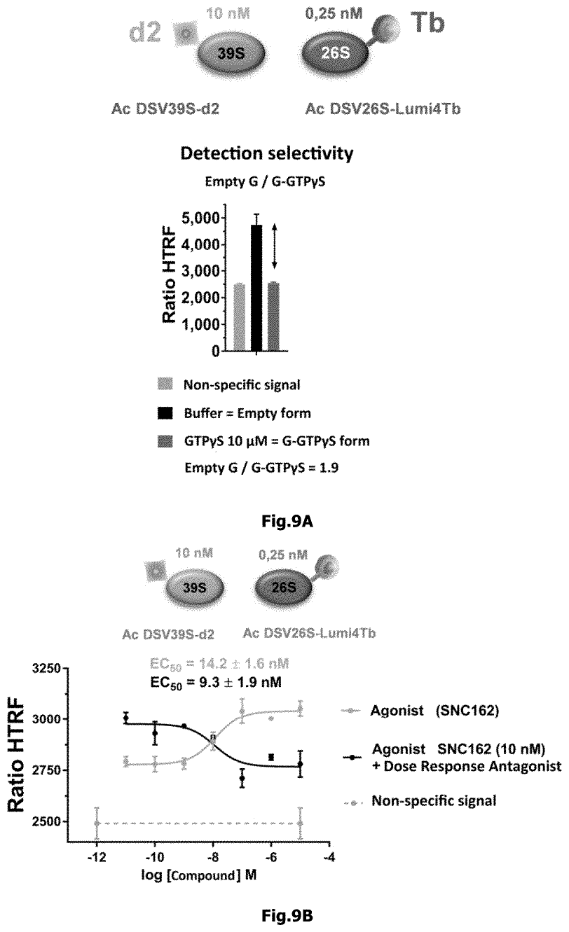

First, the ability of the detection ligand pair to specifically detect the empty Galpha protein form versus the GDP-bound full Galpha protein form was demonstrated using HEK293 cell membranes expressing the Delta Opioid GPCR and Galphai protein. The membranes were incubated with an excess of GDP (10 μM), allowing the G protein to be loaded with the nucleotide (full Galpha protein form bound to GDP), or with buffer alone (empty Galpha protein form). The difference in TR-FRET signal (HTRF ratio) observed between these two conditions shows that the detection ligand pair used (anti-Galphai antibody DSV39S-d2+anti-Galphai antibody DSV26S-Lumi4Tb—FIG. 8A) makes it possible to discriminate between the empty Galpha protein form and the full Galpha protein form bound to GDP.

In a second step, the ability of a GPCR agonist to modulate the proportion of G protein in empty form was tested with the same membranes and the same detection ligand pair. HEK293 cell membranes expressing Delta Opioid GPCR and Galphai protein were incubated with GDP (10 μM) and GPCR agonist (SNC162) in increasing concentration. The increase in the TR-FRET signal (HTRF ratio) generated by stimulation with the agonist means that the proportion of G protein in empty form increases. Thus, the GPCR receptor activated by its agonist causes the GDP of the G protein to exit the GDP, which then passes into empty form and increases the TR-FRET signal. In a second condition, activation with a fixed concentration of GPCR agonist SNC162 (10 nM) was inhibited by an increasing concentration of GPCR antagonist (Naltrindole). This activation inhibition is observed by the decrease in the TR-FRET signal (HTRF ratio). These results are shown in FIG. 8B.

### EXAMPLE 6

### Activation Test According to Format 2A: Discrimination of the Empty Form of the GTP or GTPγS Form) with a Detection Ligand Pair Labeled with TR-FRET Partners

First, the ability of the detection ligand pair to specifically detect the empty Galpha protein form against the full Galpha protein form bound to GTP or bound to GTPγS was demonstrated using HEK293 cell membranes expressing the Delta Opioid GPCR and Galphai protein. The membranes were incubated with an excess of GTPγS (10 μM), allowing the G protein to be loaded with the nucleotide (full Galpha protein form bound to GTPγS), or with buffer alone (empty Galpha protein form). The difference in TR-FRET signal (HTRF ratio) observed between these two conditions shows that the detection ligand pair used (anti-Galphai antibody DSV39S-d2+anti-Galphai antibody DSV26S-Lumi4Tb—FIG. 9A) makes it possible to distinguish the empty Galpha protein form from the full Galpha protein form bound to GTPγS

In a second step, the ability of a GPCR agonist to modulate the proportion of G protein in empty form was tested with the same membranes and the same detection ligand pair. HEK293 cell membranes expressing Delta Opioid GPCR and Galphai protein were incubated with GTPγS (10 nM) and GPCR agonist (SNC162) in increasing concentration. The increase in the TR-FRET signal (HTRF ratio) generated by stimulation with the agonist means that the proportion of G protein in empty form increases. Thus, the GPCR receptor activated by its agonist causes the GTPgS to exit the G protein which then passes into empty form and causes the TR-FRET signal to increase. In a second condition, activation with a fixed concentration of agonist SNC162 (10 nM) is inhibited by an increasing concentration of GPCR antagonist (Naltrindole). This activation inhibition is observed by the decrease in the TR-FRET signal (HTRF ratio). These results are shown in FIG. 9B.

### Example 7

### Validation of the Activation Test on Different GPCRs and Cell Lines (on Format 2B: Discrimination of the Full Galpha Protein Form Bound to GTP or Bound to GTPγS from the Empty Galpha Protein Form with the Detection Ligand Pair Labeled with TR-FRET Partners: Peptide KB1753-biotine/SA-XL/Anti-Galphai Antibody SC13533-Lumi4Tb)

The principle of activation detection was validated on four different GPCRs (Delta Opioid, GalaninR1, NOP, 5HT1A) and on two different cell lines (HEK293 and CHO-K1).

On the one hand, membranes (10 μg/wells) of HEK293 cells expressing the GPCR Delta Opioid or GALR1 (Galanin receptor) or NOP (Nociceptin receptor) or 5HT1A and the Galphai protein were incubated with GTPγS (100 nM) alone or in combination with a saturation fixed concentration (1 μM) of the receptor agonist (SNC162, Galanin, Nociceptin and serotonin respectively). For the four GPCRs, the decrease in the TR-FRET signal (HTRF ratio) generated by stimulation with the receptor agonist means that the proportion of G protein in empty form increases. Thus, the GPCR receptor activated by its agonist causes the GTPgS to exit the G protein which then passes into empty form and causes the TR-FRET signal to decrease. The differences in signals from one receptor to another on the condition of membranes incubated with GTPγS alone can be explained by differences in the expression levels of Galphai proteins in the different membrane preparations. Similarly, differences in signal modulation amplitude observed between the different GPCRs may be due to differences in expression levels of Galphai proteins or GPCRs.

On the other hand, membranes (10 μg/wells) of HEK293 or CHO-K1 cells expressing the Delta Opioid GPCR and Galphai protein were incubated with GTPγS (100 nM) alone or in combination with a saturated fixed concentration (1 μM) of the receptor agonist (SNC162). For both cell lines, the decrease in the TR-FRET signal (HTRF ratio) generated by stimulation with the agonist means that the proportion of G protein in empty form increases. Thus, the GPCR receptor activated by its agonist in turn activates the G protein which then passes in empty form and causes the TR-FRET signal to decrease.

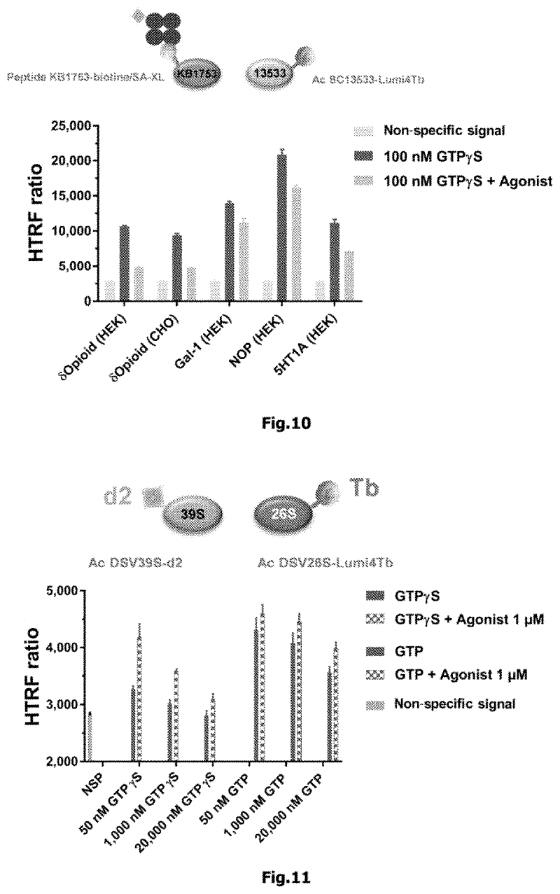

These results are shown in FIG. 10.

### EXAMPLE 8

### Comparison of GTPγS and GTP for the Activation Test According to Format 2A (Discrimination of the Empty Galpha Protein Form from the Full Galpha Protein Form Bound to GTP or Bound to GTPγS) with the Detection Ligand Pair: Anti-Galphai Antibody DSV39S-d2+Anti-Galphai Antibody DSV26S-Lumi4Tb

Membranes (1 μg/well) of HEK293 cells expressing Delta Opioid GPCR and Galphai protein were incubated with GTPγS or GTP (50, 1000 or 20000 nM) alone or in combination with a saturated fixed concentration (1 μM) of GPCR agonist (SNC162). For both nucleotides, GTPγS and GTP, the increase in the TR-FRET signal (HTRF Ratio) generated by stimulation with the agonist means that the proportion of G protein in empty form increases. Thus, the GPCR receptor activated by its agonist causes the GTP or GTPgS to exit the G protein, which then passes into empty form and causes the TR-FRET signal to increase. If both conditions (GTPγS and GTP) work to detect activation of GPCR by the agonist, the signal modulation amplitude observed with GTPγS is significantly better than with GTP. These differences in signal modulation amplitude can be explained by instability of the full Galpha protein form bound to GTP (i.e. hydrolysis of GTP to GDP).

These results are shown in FIG. 11.

### EXAMPLE 9

### Activation Test According to Format 2B: Discrimination of the Full Galphai Form (GTP or GTPγS) of the Empty Galphai Form with a Pair of Antibodies Labeled with TR-FRET Partners (Anti-Galphai Antibody SC13533-d2/Anti-Galphai Antibody DSV36S-Lumi4Tb)

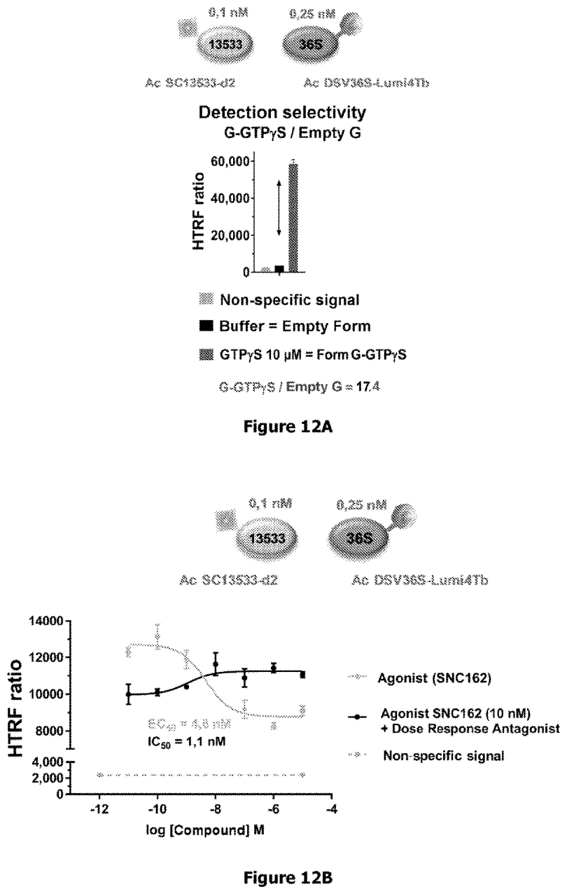

First, the ability of detection ligand pairs to specifically detect full Galpha protein bound to GTP or bound to GTPγS against empty Galpha protein was demonstrated using HEK293 cell membranes expressing the Delta Opioid GPCR and Galphai protein. The membranes were incubated with an excess of GTPγS (10 μM), allowing the Galpha protein to be loaded with the nucleotide (full Galpha protein form bound to GTPγS), or with buffer alone (empty Galpha protein form). The difference in TR-FRET signal (HTRF ratio) observed between these two conditions shows that the detection ligand pair used (anti-Galphai DSV36S-Lumi4Tb/anti-Galphai SC13533-d2) can discriminate between the Galpha protein form bound to GTPyS and the empty Galpha protein form (FIG. 12A).

In a second step, the ability of a GPCR agonist to modulate the proportion of Galpha protein in empty form was tested with the same membranes and detection ligand pairs.

In a first condition, HEK293 cell membranes expressing Delta Opioid GPCR and Galphai protein were incubated with GTPγS (10 nM) and a GPCR agonist (SNC162) in increasing concentration. A decrease in the TR-FRET signal (HTRF ratio) generated by stimulation with the agonist means that the proportion of empty Galpha protein form increases. Thus, the GPCR receptor activated by its agonist causes the GTPgS to exit from the G protein, which then passes into its empty form and decreases the TR-FRET signal.

In a second condition, HEK293 cell membranes expressing Delta Opioid GPCR and Galphai protein were incubated with GTPγS (10 nM), a fixed concentration of a SNC162 GPCR agonist (10 nM) and an increasing concentration of a GPCR antagonist (Naltrindole). Activation by the agonist is therefore inhibited when the concentration of the antagonist increases. This activation inhibition was observed by the increase in the TR-FRET signal (HTRF ratio).

The results are shown in FIG. 12B.

### EXAMPLES 10

### Activation Test According to Format 2B: Discrimination of the Full Galphai Form (GTP or GTPγS) of the Empty Galphai Form with Ligand Pairs Labeled with TR-FRET Partners.

Eight ligand pairs were used:
anti-Galphai sdAb F11 (SEQ ID NO: 2)/anti-Galphai antibody SC13533;
anti-Galphai sdAb F4 (SEQ ID NO: 3)/anti-Galphai antibody SC13533;
anti-Galphai sdAb G6 (SEQ ID NO: 4)/anti-Galphai antibody SC13533;
anti-Galphai sdAb B11 (SEQ ID NO: 5)/anti-Galphai antibody SC13533;
anti-Galphai sdAb F9 (SEQ ID NO: 6)/anti-Galphai antibody SC13533;
anti-Galphai sdAb G7 (SEQ ID NO: 7)/anti-Galphai antibody SC13533;
anti-Galphai sdAb A10 (SEQ ID NO: 8)/anti-Galphai antibody DSV36S;
anti-Galphai sdAb E2 (SEQ ID NO: 9)/anti-Galphai antibody SC13533.

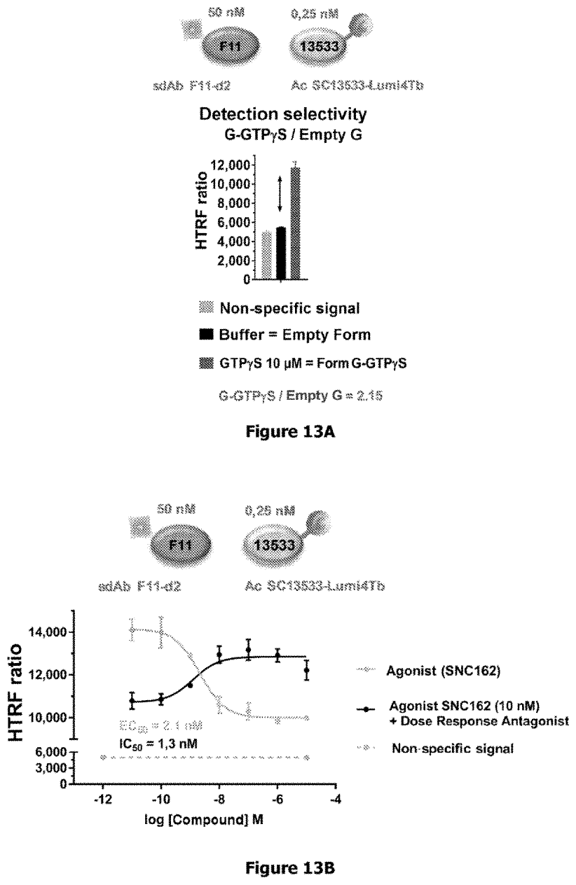

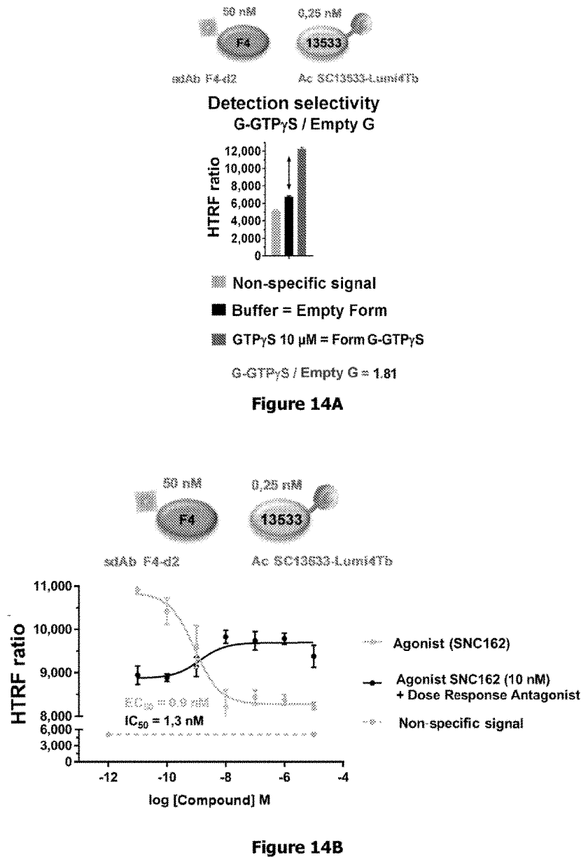

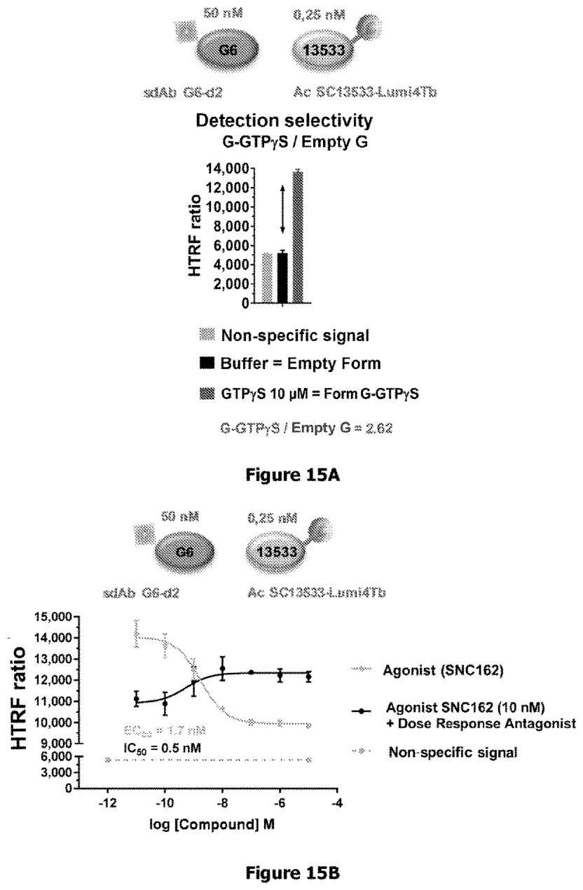

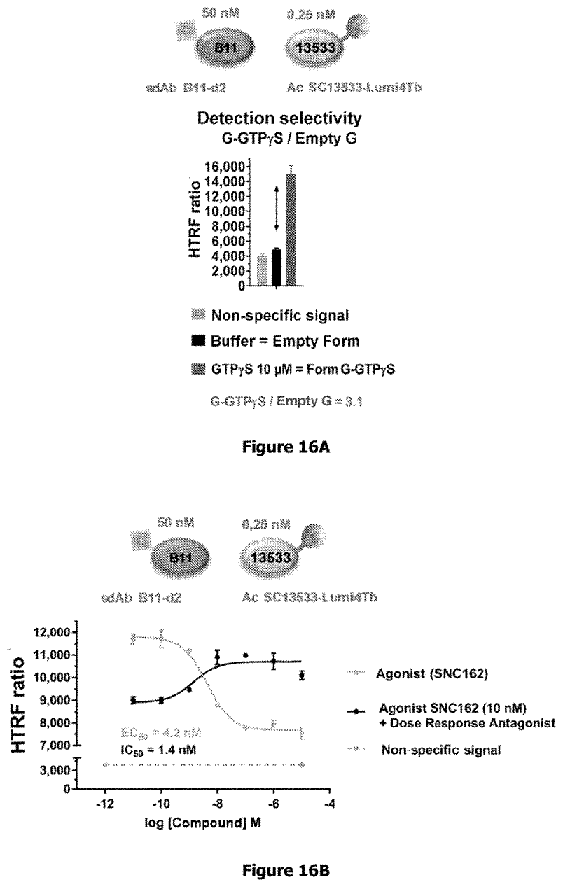

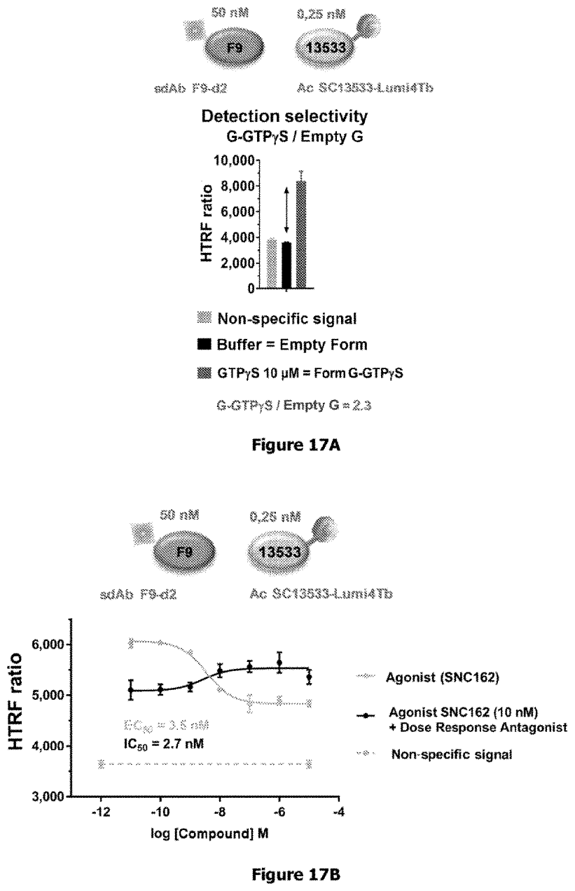

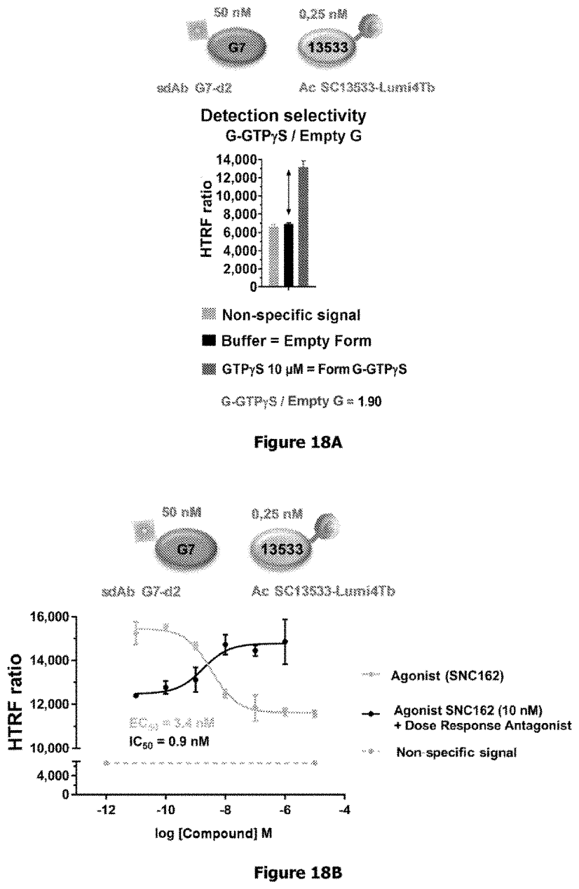

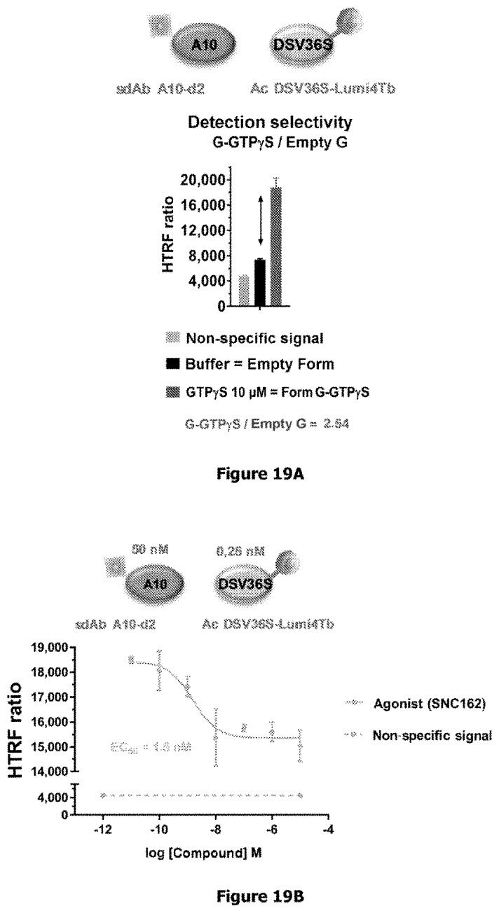

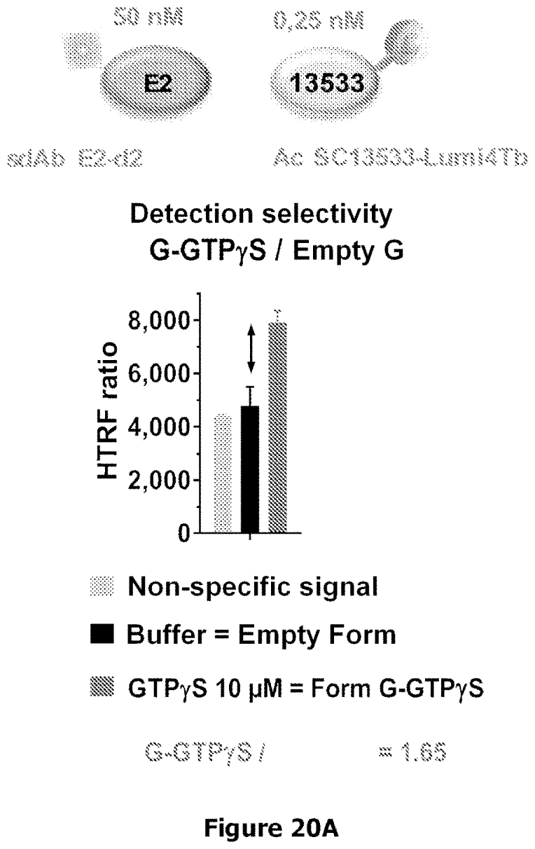

First, the ability of ligand pairs to specifically detect the full Galphai protein bound to GTP or bound to GTPγS against the empty Galphai protein was demonstrated using HEK293 cell membranes expressing the Delta Opioid GPCR and Galphai protein. The membranes were incubated with an excess of GTPγS (10 μM), allowing the Galpha protein to be loaded with the nucleotide (full Galpha protein form bound to GTPγS), or with buffer alone (empty Galpha protein form). The difference in TR-FRET signal (HTRF ratio) observed between these two conditions shows that the 8 detection ligand pairs used make it possible to distinguish the full Galpha protein form bound to GTPγS from the empty Galpha protein form (anti-Galphai sdAb F11-d2/anti-Galphai SC13533-Lumi4Tb antibody—FIG. 13A; anti-Galphai sdAb F4-d2/anti-Galphai SC13533-Lumi4Tb antibody—FIG. 14A; anti-Galphai sdAb G6-d2/anti-Galphai antibody SC13533-Lumi4Tb — FIG. 15A; anti-Galphai sdAb B11-d2/anti-Galphai antibody SC13533-Lumi4Tb—FIG. 16A; anti-Galphai sdAb F9-d2/anti-Galphai antibody SC13533-Lumi4Tb—FIG. 17A; anti-Galphai sdAb G7-d2/anti-Galphai antibody SC13533-Lumi4Tb—FIG. 18A; anti-Galphai sdAb A10-d2/anti-Galphai antibody DSV36S-Lumi4Tb—FIG. 19A; anti-Galphai sdAb E2-d2/anti-Galphai antibody SC13533-Lumi4Tb—FIG. 20A).

In a second step, the ability of a GPCR agonist to modulate the activation of a GPCR, by measuring the proportion of Galpha protein in empty form, was tested with HEK293 cell membranes expressing Delta Opioid GPCR and Galphai protein and the eight ligand pairs mentioned above.

In a first condition, the membranes were incubated with GTPγS (10 nM) and a GPCR agonist (SNC162) in increasing concentration. The decrease in the TR-FRET signal (HTRF ratio) generated by stimulation with the agonist means that the proportion of empty Galpha protein form increases. The GPCR receptor activated by its agonist causes the GTPgS to exit from the Galpha protein, which then passes into its empty form and causes the TR-FRET signal to decrease.

In a second condition, the membranes were incubated with GTPγS (10 nM), a fixed concentration of an GPCR agonist SNC162 (10 nM) and an increasing concentration of a GPCR antagonist (Naltrindole). Activation by the agonist is therefore inhibited when the concentration of the antagonist increases. This activation inhibition was observed by the increase in the TR-FRET signal (HTRF ratio).

The results are shown in FIGS. 13B, 14B, 15B, 16B, 17B, 18B and 19B (The second condition was not implemented in FIG. 19B).

The content of the ASCII text file of the sequence listing named “Substitute-Sequence-Listing-26May2020-21721-0801”, having a size of 11.1 kb and a creation date of 26 May 2020, and electronically submitted via EFS-Web on 26 May 2020, is incorporated herein by reference in its entirety.

1. A method for determining the ability of a molecule to modulate the activation of a G-protein-coupled receptor (GPCR), said method comprising the following steps:
a) introduction, into a first container, of:
a membrane preparation carrying one or more GPCRs and one or more G proteins,
a GDP source or a GTP source,
a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
b) measurement of the RET signal emitted in the first container;
c) introduction (i) in a second container, of the same reagents as in step a) and of the test molecule or (ii) in the first container, of the test molecule;
d) measurement of the RET signal emitted in the second container or in the first container obtained in step c);
e) comparison of the signals obtained in steps b) and d);
an increase in signal when using a GDP source and ligands capable of binding specifically to the empty Galpha protein indicating that the molecule is a GPCR agonist;
a decrease in signal when using a GDP source and ligands capable of binding specifically to the empty Galpha protein indicating that the molecule is a GPCR inverse agonist;
a decrease in signal when using a GDP source and ligands capable of binding specifically to the full Galpha protein indicating that the molecule is a GPCR agonist;
an increase in signal when using a GDP source and ligands capable of binding specifically to the full Galpha protein indicating that the molecule is a GPCR inverse agonist;
an increase in signal when using a GTP source and ligands capable of binding specifically to the empty Galpha protein indicating that the molecule is a GPCR agonist;
a decrease in signal when using a GTP source and ligands capable of binding specifically to the empty Galpha protein indicating that the molecule is a GPCR inverse agonist;
a decrease in signal when using a GTP source and ligands capable of binding specifically to the full Galpha protein indicating that the molecule is a GPCR agonist;
an increase in signal when using a GTP source and ligands capable of binding specifically to the full Galpha protein indicating that the molecule is a GPCR inverse agonist.

2. A method for determining the ability of a molecule to modulate the activation of a G-protein-coupled receptor (GPCR), said method comprising the following steps:
a) introduction, into a first container, of:
a membrane preparation carrying one or more GPCRs and one or more G proteins,
a GDP source or a GTP source,
a first ligand of the G protein alpha subunit (Galpha protein) labeled with a first member of a RET partner pair, and a second ligand of the Galpha protein labeled with a second member of the RET partner pair, said ligands being capable of binding specifically, either individually or in combination, to the empty Galpha protein or to the full Galpha protein;
a GPCR agonist;
b) measurement of the RET signal emitted in the first container;
c) introduction into a second container of the same reagents as in step a) and of the test molecule;
d) measurement of the RET signal emitted in the second container obtained in step c);
e) comparison of the signals obtained in steps b) and d);
an increase in signal when using a GDP source and ligands capable of binding specifically to the empty Galpha protein indicating that the molecule is a GPCR agonist or positive allosteric modulator;
a decrease in signal when using a GDP source and ligands capable of binding specifically to the full Galpha protein indicating that the molecule is a GPCR agonist or positive allosteric modulator;
an increase in signal when using a GTP source and ligands capable of binding specifically to the empty Galpha protein indicating that the molecule is a GPCR agonist or positive allosteric modulator;
a decrease in signal when using a GTP source and ligands capable of binding specifically to the full Galpha protein indicating that the molecule is a GPCR agonist or positive allosteric modulator.
a decrease in signal when using a GDP source and ligands capable of binding specifically to the empty Galpha protein indicating that the molecule is a GPCR antagonist or negative allosteric modulator;
an increase in signal when using a GDP source and ligands capable of binding specifically to the full Galpha protein indicating that the molecule is a GPCR antagonist or negative allosteric modulator;
a decrease in signal when using a GTP source and ligands capable of binding specifically to the empty Galpha protein indicating that the molecule is a GPCR antagonist or negative allosteric modulator;
an increase in signal when using a GTP source and ligands capable of binding specifically to the full Galpha protein indicating that the molecule is a GPCR antagonist or negative allosteric modulator.

3. The method as claimed in claim 1, wherein said Galpha protein is selected from Galphai1, Galphai2, Galphai3, Galphao1, Galphao2, Galphaq, Galpha12, Galpha13, Galphas, Galphaz, Galphat1, Galphat2, Galpha11, Galpha14, Galpha15, Galpha16 and Galphagus protein, preferably selected from Galphai1, Galphai2 and Galphai3.

4. The method as claimed in claim 1, wherein the first ligand and the second ligand are selected from an antibody, an antibody fragment, a peptide or an aptamer.

5. The method as claimed in claim 1, wherein the first ligand is a peptide and the second ligand is an antibody or antibody fragment.

6. The method as claimed in claim 1, wherein the first ligand and the second ligand are antibodies or antibody fragments.

7. The method as claimed in claim 1, wherein the first ligand is a peptide of the sequence Ser-Ser-Arg-Gly-Tyr-Tyr-His-Gly-Ile-Trp-Val-Gly-Glu-Glu-Gly-Arg-Leu-Ser-Arg (SEQ ID NO: 1) (peptide KB1753) and the second ligand is an anti-Galphai antibody.

8. The method as claimed in claim 1, wherein the first ligand is an sdAb of sequence selected from SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 and the second ligand is an anti-Galphai antibody.

9. The method as claimed in claim 1, wherein the GTP source is a non-hydrolyzable or slowly hydrolyzable GTP, preferably selected from GTPgammaS (GTPγS), GppNHp and GppCp.

10. The method as claimed in claim 1, wherein the first ligand and the second ligand are each labeled with a member of a RET partner pair, one of the members of the pair being a luminescent donor compound or a fluorescent donor compound and the other member of the pair being a fluorescent acceptor compound or a non-fluorescent acceptor compound (quencher).

11. The method as claimed in claim 1, wherein the first ligand and the second ligand are each labeled with a member of a RET partner pair, one of the members of the pair being a fluorescent donor compound and the other member of the pair being a fluorescent acceptor compound or a non-fluorescent acceptor compound (quencher), and wherein said fluorescent donor compound is a FRET partner selected from: europium cryptate, europium chelate, terbium chelate, terbium cryptate, ruthenium chelate, quantum dot, allophycocyanins, rhodamines, cyanins, squarains, coumarins, proflavins, acridines, fluoresceins, boron-dipyrromethene derivatives and nitrobenzoxadiazole.

12. The method as claimed in claim1, wherein the first ligand and the second ligand are each labeled with a member of a RET partner pair, one of the members of the pair being a luminescent donor compound or a fluorescent donor compound and the other member of the pair being a fluorescent acceptor compound, and wherein said fluorescent acceptor compound is a FRET partner selected from: allophycocyanins, rhodamines, cyanins, squarains, coumarins, proflavins, acridins, fluoresceins, boron-dipyrromethene derivatives, nitrobenzoxadiazole, a quantum dot, GFP, GFP variants selected from GFP10, GFP2 and eGFP, YFP, YFP variants selected from eYFP, YFP topaz, YFP citrine, YFP venus and YPet, mOrange, DsRed.

13. The method as claimed in claim 1, wherein the first ligand and the second ligand are each labeled with a member of a RET partner pair, one of the members of the pair being a luminescent donor compound and the other member of the pair being a fluorescent acceptor compound or a non-fluorescent acceptor compound (quencher), and whereinsaid luminescent donor compound is a BRET partner selected from: Luciferase (luc), Renilla Luciferase (Rluc), variants of Renilla Luciferase (Rluc8) and Firefly Luciferase.

14. The method as claimed in claim 1, wherein the first ligand and the second ligand are each labeled with a member of a RET partner pair, one of the members of the pair being a luminescent donor compound or a fluorescent donor compound and the other member of the pair being a fluorescent acceptor compound, and wherein said fluorescent acceptor compound is a BRET partner selected from: allophycocyanins, rhodamines, cyanins, squarains, coumarins, proflavins, acridins, fluoresceins, boron-dipyrromethene derivatives, nitrobenzoxadiazole, a quantum dot, GFP, GFP variants selected from GFP10, GFP2 and eGFP, YFP, YFP variants selected from eYFP, YFP topaz, YFP citrine, YFP venus and YPet, mOrange, DsRed.

15. A kit for implementing the method as claimed in claim 1, comprising:
a first ligand of the G protein alpha subunit (Galpha protein) labeled with a first member of a RET partner pair and a second ligand of the Galpha protein labeled with a second member of the RET partner pair, said ligands being capable of binding specifically, either individually or in combination, to the empty Galpha protein or to the full Galpha protein;
a set of instructions;
optionally one or more dilution buffers for the reagents;
optionally a GDP source or a GTP source;
optionally a membrane preparation carrying one or more GPCRs and/or one or more proteins; and
optionally a Galpha protein.

16. The method as claimed in claim 2, wherein said Galpha protein is selected from Galphai1, Galphai2, Galphai3, Galphao1, Galphao2, Galphaq, Galpha12, Galpha13, Galphas, Galphaz, Galphat1, Galphat2, Galpha11, Galpha14, Galpha15, Galpha16 and Galphagus protein, preferably selected from Galphai1, Galphai2 and Galphai3.

17. The method as claimed in claim 2, wherein the first ligand and the second ligand are selected from an antibody, an antibody fragment, a peptide or an aptamer.

18. The method as claimed in claim 2, wherein the first ligand is a peptide and the second ligand is an antibody or antibody fragment.

19. The method as claimed in claim 2, wherein the first ligand and the second ligand are antibodies or antibody fragments.

20. The method as claimed in claim 2, wherein the first ligand is a peptide of the sequence Ser-Ser-Arg-Gly-Tyr-Tyr-His-Gly-Ile-Trp-Val-Gly-Glu-Glu-Gly-Arg-Leu-Ser-Arg (SEQ ID NO: 1) (peptide KB1753) and the second ligand is an anti-Galphai antibody.

21. The method as claimed in claim 2, wherein the first ligand is an sdAb of sequence selected from SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 and the second ligand is an anti-Galphai antibody.

22. The method as claimed in claim 2, wherein the GTP source is a non-hydrolyzable or slowly hydrolyzable GTP, preferably selected from GTPgammaS (GTPγS), GppNHp and GppCp.

23. The method as claimed in claim 2, wherein the first ligand and the second ligand are each labeled with a member of a RET partner pair, one of the members of the pair being a luminescent donor compound or a fluorescent donor compound and the other member of the pair being a fluorescent acceptor compound or a non-fluorescent acceptor compound (quencher).

24. The method as claimed in claim 2, wherein the first ligand and the second ligand are each labeled with a member of a RET partner pair, one of the members of the pair being a fluorescent donor compound and the other member of the pair being a fluorescent acceptor compound or a non-fluorescent acceptor compound (quencher), said fluorescent donor compound is a FRET partner selected from: europium cryptate, europium chelate, terbium chelate, terbium cryptate, ruthenium chelate, quantum dot, allophycocyanins, rhodamines, cyanins, squarains, coumarins, proflavins, acridines, fluoresceins, boron-dipyrromethene derivatives and nitrobenzoxadiazole.

25. The method as claimed in claim 2, wherein the first ligand and the second ligand are each labeled with a member of a RET partner pair, one of the members of the pair being a luminescent donor compound or a fluorescent donor compound and the other member of the pair being a fluorescent acceptor compound, said fluorescent acceptor compound is a FRET partner selected from: allophycocyanins, rhodamines, cyanins, squarains, coumarins, proflavins, acridins, fluoresceins, boron-dipyrromethene derivatives, nitrobenzoxadiazole, a quantum dot, GFP, GFP variants selected from GFP10, GFP2 and eGFP, YFP, YFP variants selected from eYFP, YFP topaz, YFP citrine, YFP venus and YPet, mOrange, DsRed.

26. The method as claimed in claim 2, wherein the first ligand and the second ligand are each labeled with a member of a RET partner pair, one of the members of the pair being a luminescent donor compound and the other member of the pair being a fluorescent acceptor compound or a non-fluorescent acceptor compound (quencher), said luminescent donor compound is a BRET partner selected from: Luciferase (luc), Renilla Luciferase (Rluc), variants of Renilla Luciferase (Rluc8) and Firefly Luciferase.

27. The method as claimed in claim 2, wherein the first ligand and the second ligand are each labeled with a member of a RET partner pair, one of the members of the pair being a luminescent donor compound or a fluorescent donor compound and the other member of the pair being a fluorescent acceptor compound, said fluorescent acceptor compound is a BRET partner selected from: allophycocyanins, rhodamines, cyanins, squarains, coumarins, proflavins, acridins, fluoresceins, boron-dipyrromethene derivatives, nitrobenzoxadiazole, a quantum dot, GFP, GFP variants selected from GFP10, GFP2 and eGFP, YFP, YFP variants selected from eYFP, YFP topaz, YFP citrine, YFP venus and YPet, mOrange, DsRed.

28. A kit for implementing the method as claimed in claim 2 comprising:
a first ligand of the G protein alpha subunit (Galpha protein) labeled with a first member of a RET partner pair and a second ligand of the Galpha protein labeled with a second member of the RET partner pair, said ligands being capable of binding specifically, either individually or in combination, to the empty Galpha protein or to the full Galpha protein;
a set of instructions;
optionally one or more dilution buffers for the reagents;
optionally a GDP source or a GTP source;
optionally a membrane preparation carrying one or more GPCRs and/or one or more proteins; and
optionally a Galpha protein.
